(19) United States
(12) Patent Application Publication
(10) Pub. No.: US 2026/0026509 A1
(43) Pub. Date: Jan. 29, 2026

(54) BIOLOGICAL PLANT PROTECTION AGENT, METHODS AND USES THEREOF

(71) Applicant: INNOVPLANTPROTECT – ASSOCIAÇÃO, ELVAS (PT)

(72) Inventors: Cristina Maria LOURENÇO DA CUNHA CORREIA DE AZEVEDO, ELVAS (PT); Margarida BASALOCO FONSECA, ELVAS (PT); Pedro CARRANCA NUNES ROSA, ELVAS (PT); Tiago Miguel MARQUES MONTEIRO AMARO, ELVAS (PT); Fátima Alexandra MEIRA MACHADO, ELVAS (PT); David Alexander LEARMONTH, ELVAS (PT)

(73) Assignee: INNOVPLANTPROTECT – ASSOCIAÇÃO, ELVAS (PT)

(21) Appl. No.: 18/995,526

(22) PCT Filed: Jul. 21, 2023
(86) PCT No.: PCT/IB2023/057464

§ 371 Date: Jan. 16, 2025

(30) Foreign Application Priority Data
Jul. 21, 2022 (PT) 118117
Jul. 20, 2023 (PT) 118827

(51) Int. Cl. A01N 63/22; A01P 1/00; A01P 3/00; C12N 1/20; C12R 1/07

(52) U.S. Cl. CPC A01N 63/22; A01P 1/00; A01P 3/00; C12N 1/205; C12R 2001/07

(57) ABSTRACT

The present disclosure relates to a Bacillus velezensis strain, and a composition for plant protection comprising an effective amount of said Bacillus velezensis. The disclosed Bacillus velezensis strain or composition can be used as a biological plant protection agent. A method for protecting or treating plants and/or fruit from fungal or bacterial infections comprising applying an effective amount of the Bacillus velezensis strain or the composition is also disclosed.

### TECHNICAL FIELD

The present disclosure relates to the field of biological plant protection agents (henceforth termed BPPAs), more specifically to a strain of Bacillus velezensis (henceforth termed B. velezensis), its formulations, uses and methods for the control of plant diseases, in particular fire blight in fruit plants, pome fruits and ornamental plants, or rice-blast.

### BACKGROUND

Agriculture involves cultivation of plants for food, biofuels and raw materials. Crops are endangered by pests and diseases which can drastically reduce harvest yields. Pesticides are substances that are able to repel, eradicate or mitigate pests and diseases. According to the Food and Agriculture Organisation of the United Nations, four million tonnes of chemical-based pesticides were used globally in 2018, causing negative impact on soil, air and water quality as well as affecting non-target organisms.

Fire blight is a particularly devastating systemic necrotic plant disease affecting apple and pear trees as well as many other plant members of the Rosaceae family. Disease symptoms on the plant are characterised by scorched appearance, necrosis and wilting and bacterial ooze. Fire blight is caused by the pathogen Erwinia amylovora (henceforth termed E. amylovora), a facultative anaerobe and non-sporulating, rod-shaped Gram-negative bacterium that belongs to the Enterobacteriaceae family. Fire blight is believed to have originated in North America in the late 18th century but has since spread worldwide as it is readily transmitted by wind, insects, rain, pruning and asymptomatic host plants. Primary infection typically occurs in the epiphytic phase in flowers, in which the pathogen begins to multiply and enters into contact with wounds or natural openings like stomata. Eventually, colonised tissues in trunks or branches evolve to overwintering cankers. Once fire blight is established in the plant, immediate action is required to segregate the infected portion of the plant by extensive pruning. Delay or failure to prune in a timely manner is usually fateful, culminating in the forced destruction of entire orchards. Strains of Erwinia amylovora are also some of the most prevalent bacterial agents responsible for spoilage of fresh fruit and vegetables post-harvest. This dual attack on both plants and post-harvested fruit creates massive economic losses as well as the need for large scale destruction of millions of plants and tonnes of wasted food.

Currently, control of fire blight remains extremely challenging as there are limited commercially available solutions with antagonist properties that directly affect the pathogen responsible. Excessive use of copper salts, aluminium phosphates and chemical-based pesticides is commonplace, but outcomes are unsatisfactory and such treatments lead to pesticide accumulation over time resulting in potentially toxic levels in soils.

Furthermore, another important aspect in fire blight disease management is related to the development of microbial resistance, leading to the appearance and spread of even more potent E. amylovora strains that fail to respond to powerful antibiotics, such as streptomycin or tetracycline, whose use is permitted in some regions worldwide.

This evidence, taken together, clearly indicates that there exists a pressing urgency across the globe for efficacious, environmentally benign, sustainable, and economically viable solutions to control fire blight disease caused by E. amylovora.

Consequently, several BPPAs have emerged in the patent literature that are claimed to be able to control E. amylovora. Among these BPPAs are phages (Mazzuchi, 2014), essential oils (Mullen, 2011) and recombinant peptides (Gupta, 2021). The most prevalent class though are purified single bacterial strains, such as, for example, Lactobacillus plantarum (Umemura, 2022), Erwinia herbicola (Pusey, 1999), Pantonea agglomerans (Dagher, 2022), Pseudomonas fluorescens (Armstrong, 2010), Bacillus subtilis (Heins, 2000) or even multi-bacterial strain concoctions (Brower, 2011).

However, to date, no cure or truly efficacious standalone BPPA with target specificity for E. amylovora has become available for widespread use by the agricultural community.

Another disease of concern is rice blast in rice plants. Rice is a major staple food crop for more than 3.5 billion people around the globe and provides 20% of all the calories consumed by the world population (Elert, 2014). The worldwide population is expected to grow up to 9.8 billion by the end of 2050 and thus food demand will greatly increase, wherein rice crops will certainly be a key player. Rice growers though will face a huge challenge to increase rice yield by 25% to ensure food availability (Skamnioti, 2009). On the other hand, it is estimated that crop losses due to rice blast disease deprives 60 million people of food (Pennisi, 2010).

Rice blast is caused by the filamentous fungus Magnaporthe oryzae (anamorph Pyricularia oryzae, henceforth referred to as M. orzyae), a plant pathogenic fungus. The pathogen infects as a spore that produces lesions or spots on parts of the rice plant such as the leaf, leaf collar, panicle, culm and culm nodes. Using a structure called an appressorium, the pathogen is able to penetrate the plant tissues. Initial symptoms are white to grey-green lesions or spots with darker borders produced on all parts of the shoot, while older lesions are elliptical or spindle-shaped and whitish to gray with necrotic borders. Lesions may enlarge and coalesce to kill the entire leaf and panicles followed by significant crop loss in total production. Symptoms are observed on all above-ground parts of the plant.

Currently employed chemical-based fungicides against M. orzyae are generally ineffective due in part to the fungus acquired resistance and are also environmentally toxic and to date, no cure or truly efficacious standalone sustainable bioprotectant with target specificity against M. orzyae has become available for widespread use by the agricultural community.

These facts are disclosed in order to illustrate the technical problem addressed by the present disclosure.

### General Description

The present disclosure relates to the field of BPPAs, more specifically to a new strain of B. velezensis, its formulations, uses and methods for the control of plant diseases, in particular fire blight in fruit plants, pome fruits and ornamental plants, or rice blast in rice plants.

Several B. velezensis strains have been reported to suppress the growth of pathogens, including bacteria, fungi and nematodes. Genomic analysis revealed that B. velezensis possesses strain-specific clusters of genes related to the biosynthesis of certain secondary metabolites, which play significant roles in both pathogen suppression and plant growth promotion. B. velezensis is capable of the biosynthesis of complex metabolites including cyclic lipopeptides such as surfactin, bacillomycin-D, fengycin and bacillibactin, as well as polyketides such as macrolactin, bacillaene and difficidin (Chen, 2007).

Indeed, several strains of B. velezensis have been recently proposed as ecologically safe BPPAs as well as plant growth promoters (Fan, 2018, Reva, 2019, Joly, 2021, Alenezi, 2021, Gharsa, 2021, Myo, 2021, Shin, 2021, Kim, 2021, Wang, 2022).

In particular, the present disclosure relates to an isolated B. velezensis strain, with the deposit with number CECT30902 received on 12 Jul. 2023 at the Spanish Type Culture Collection (CECT) (henceforth termed ‘I21’ or ‘InPP (I21)’) that has proven to be surprisingly efficacious against the causal agent of fire blight, E. amylovora in comparison to more than 200 bacterial endophytes isolated from different plant species (namely pear, almond, olive amongst others) that were also screened but found ineffective, as well as a commercial BPPA containing another Bacillus strain, namely Bacillus subtilis QST 713 (Serenade ASO®), henceforth also termed ‘Commercial (I16)’. The 21 strain was isolated from pear plants of the Rocha variety from an orchard located in the Oeste region of Portugal. The I21 strain was selected for further testing based on its unexpectedly strong in vitro growth inhibition of a virulent E. amylovora strain (isolated from Pyrus communis cv. ‘Rocha’ from Campo Maior region in Portugal, that is henceforth termed ‘CM1’).

To genetically characterise 121, the genomic sequence of three genes were amplified and sequenced with the following primers: (1) SEQ ID No 21-16S rRNA partial sequence-16SFw AGAGTTTGATC(A:C)TGGCTCAG and SEQ ID No 22-16SRev TACGG(C:T)TACCTTGTTACGACTT (Jensen, 2002); (2) SEQ ID No 23-beta subunit of RNA polymerase (rpoB) partial sequence-rpoBFw, GACGTGGGATGGCTACAACT and SEQ ID No 24-rpoBRev, ATTGTCGCCTTTAACGATGG (Rooney, 2009); and (3) SEQ ID No 25-gyrase A (gyrA) partial sequence-gyrAFw, CAGTCAGGAAATGCGTACGTCCTT and SEQ ID No 26-gyrARev, CAAGGTAATGCTCCAGGCATTGCT (Rooney, 2009). The sequences obtained were the following:

I21 16S rRNA partial gene
SEQ ID No. 27:
ccattctgtcaccattcggcggctggctcataaaggttacctcaccgact
tcgggtgttacaaactctcgtggtgtgacgggcggtgtgtacaaggcccg
ggaacgtattcaccgcggcatgctgatccgcgattactagcgattccagc
ttcacgcagtcgagttgcagactgcgatccgaactgagaacagatttgtg
ggattggcttaacctcgcggtttcgctgccctttgttctgtccattgtag
cacgtgtgtagcccaggtcataaggggcatgatgatttgacgtcatcccc
accttcctccggtttgtcaccggcagtcaccttagagtgcccaactgaat
gctggcaactaagatcaagggttgcgctcgttgcgggacttaacccaaca
tctcacgacacgagctgacgacaaccatgcaccacctgtcactctgcccc
cgaaggggacgtcctatctctaggattgtcagaggatgtcaagacctggt
aaggttcttcgcgttgcttcgaattaaaccacatgctccaccgcttgtgc
gggcccccgtcaattcctttgagtttcagtcttgcgaccgtactccccag
gcggagtgcttaatgcgttagctgcagcactaaggggcggaaacccccta
acacttagcactcatcgtttacggcgtggactaccagggtatctaatcct
gttcgctccccacgctttcgctcctcagcgtcagttacagaccagagagt
cgccttcgccactggtgttcctccacatctctacgcatttcaccgctaca
cgtggaattccactctcctcttctgcactcaagttccccagtttccaatg
accctccccggttgagccgggggctttcacatcagacttaagaaaccgcc
tgcgagccctttacgcccaataattccggacaacgcttgccacctacgta
ttaccgcggctgctggcacgtagttagccgtggctttctggttaggtacc
gtcaaggtgccgccctatttgaacggcacttgttcttccctaacaacaga
gctttacgatccgaaaaccttcatcactcacgcggcgttgctccgtcaga
ctttcgtccattgcgganaattccctactgctgcctcccgtaggagtctg
ggccgggtctcagtcccatg
I21 rpoB partial gene
SEQ ID No. 28:
Tgacgccttgtgaagatgatgtatacacatctattcacattgaagaatat
gaatcagaagcacgtgatacaaagcttgggcctgaagagatcacccgcga
tattccaaacgtaggggaagacgcgcttcgcaatcttgatgaccgcggaa
ttatccgtatcggtgcggaagtcaacgacggagaccttctcgtaggtaaa
gtaacgcctaaaggtgtaactgagcttacggctgaagaacgccttctgca
tgcgatctttggagaaaaagcgcgtgaagtccgtgatacttctctccgtg
tgcctcacggcggcggcggaattatccacgacgtaaaagtcttcaaccgt
gaagacggcgacgaacttcctccgggagtgaaccagcttgtacgcgtata
tatcgttcagaaacgtaagatttctgaaggtgataaaatggccggacgtc
acggaaacaaaggggttatctcgaagattcttcctgaagaagatatgcct
taccttcctgacggcacgccgatcgatatcatgcttaacccgctgggtgt
accatcacgtatgaatatcggtcaggtattagaacttcacatgggtatgg
ctgcccgctacctcggcattcacatcgcgtcacctgtatttgacggcgcg
cgtgaagaagatgtgtgggaaacacttgaagaagcaggcatgtcaagaga
cgctaaaacagttctttatgacggccgtacgggagaaccgtttgacaacc
gtgtatctgtcggaatcatgtacatgatcaaactggcgcacatggttgat
gataaacttcatgcccgttctacaggtccttactcacttgttacgcagca
gcctctcggcggtaaagcccaattcggcggacagcgtttcggtgagatgg
aggtttgggcgctcgaagcttacggcgcagcttacacgcttcaagaaatc
ctgactgtgaagtccgatgacgtggtcggacgtgtgaaaacatatgaagc
ctcgtataagggggaaacaataaagg.
I21 gyrA partial gene
SEQ ID No. 29:
atcccgggcgcttccggatgtgcgtgacggtgtgaggccggttcccttga
gattttgtacgcaatgaatgatttaggcatgaccagtgacaaaccatata
aaaaatctgcccgtatcgtcggtgaagttatcggtaagtaccacccgcac
ggtgactcagcggtttacgaatcaatggtcagaatggcgcaggattttaa
ctaccgctacatgcttgttgacggacacggcaacttcggttcggttgacg
gcgactcagcggccgcgatgcgttacacagaagcgagaatgtcaaaaatc
gcaatggaaatcctccgggacattacgaaagatacgattgattatcaaga
taactatgacggcgcagaaagagaacctgtcgtcatgccttcgagatttc
cgaatctgctcgtaaacggagctgccggtattgcggtcggaatggcgaca
aatattcctccgcatcagcttggggaagtcattgaaggcgtgcttgccgt
aagtgagaatcctgagattacaaaccaggagctgatggaatacatcccgg
gcccggattttccgactgcaggtcagattttgggccggagcggcatccgc
aaggcatatgaatccggacggggatccattacgatccgggctaaggctga
aatcgaagagacatcatcgggaaaagaaagaattattgtcacagaacttc
cttatcaggtgaacaaagcgagattaattgaaaaaatcgcagatcttgtc
cgggacaaaaaaatcgaaggaattaccgatctgcgtgacgaatccgaccg
taacggaatgagaatcgtcattgagatccgccgtgacgccaatgctcacg
tcattttgaataacctgtacaaacaaacggccctgcagacgtctttcgga
atcaacctgctggcgctcgttgacggacagccgaaggtactaagcctgaa
gcaatgcctggagccattaccttgaa.

In an embodiment, phylogenetic analysis of the rpoB and gyrA gene sequences enabled a more detailed identification of 121, with high homology to Bacillus velezensis strain ZeaDK315Endobac16 for these two genes, having the GenBank accession number CP043809.1. Whole-genome sequencing of I21 (SEQ ID No. 30 to SEQ ID No. 48), followed by genome based taxonomic assignment, confirmed the strain to be a B. velezensis. A phylogenomic tree of B. velezensis, further confirmed the close relationship of I21 to ZeaDK315Endobac16.

Surprisingly, the whole-genome sequencing of strain I21, disclosed in the present document, allowed to conclude that this strain carries 21 genes (with 20 unique DNA sequences) that are unique to it (SEQ ID No. 49 to SEQ ID No. 68), relative to a collection of 489 B. velezensis genomes.

An aspect of the present disclosure relates to Bacillus velezensis strain with the deposit number CECT30902 received on 12 Jul. 2023 at the Spanish Type Culture Collection (CECT)-International Depositary Authority under the Budapest Treaty, at Colección Espanola de Cultivos Tipo.

Another aspect of the present disclosure relates to a Bacillus velezensis strain wherein the B. velezensis strain comprises the following genes SEQ ID No. 49 to SEQ ID No. 68.

Another aspect of the present disclosure relates to a Bacillus velezensis strain with a genome comprising the sequence of SEQ ID No. 30 to SEQ ID No. 48.

Another aspect of the present disclosure relates to the Bacillus velezensis of the present disclosure obtained by genetic engineering. The provided genome sequences, as well as the further characterization of the I21 strain, allow the person skilled in the art to manufacture and reproduce the strain of bacterium by utilizing synthetic biology methods, and to further verify the obtained strain. Indeed, it is known from the state of the art that it is possible to (1) use a closely related reference genome or genome collection to infer the order (scaffold) of the genome contigs (SEQ ID No. 30 to SEQ ID No. 48) (Alonge et al., 2019) (2) synthetically recreate the full genome of a new bacterium, (3) extract the natural genomic material from an existing strain of bacterium, (4) transplant into the existing bacterium (now void of its natural genome) with the synthetically created new bacterial genome, and (5) successfully culture colonies of the bacterium with the newly created synthetic genome (Pennisi, 2010 and Gibson et al., 2010).

Another aspect of the present disclosure relates to a Bacillus velezensis stain wherein the B. velezensis produces the proteins from the following list: SEQ ID No. 1, SEQ ID No. 2, SEQ ID No. 3, SEQ ID No. 4, SEQ ID No. 5, SEQ ID No. 6, SEQ ID No. 7, SEQ ID No. 8, SEQ ID No. 9, SEQ ID No. 10, SEQ ID No. 11, SEQ ID No. 12, SEQ ID No. 13, SEQ ID No. 14, SEQ ID No. 15, SEQ ID No. 16, SEQ ID No. 17, SEQ ID No. 18, SEQ ID No. 19, and SEQ ID No. 20.

In an embodiment for better results, the Bacillus velezensis of the present disclosure may be used for controlling plant or fruit diseases.

In an embodiment for better results, the Bacillus velezensis of the present disclosure may be used as a biological plant or fruit protector.

In an embodiment for better results, the disease is an infection, in particular a fungal and/or a bacterial infection.

In an embodiment for better results, the bacterial infection is caused by a gram-negative bacteria; in particular a bacteria of the genus Enterobacterales.

In an embodiment for better results, the plant is a woody plant; namely plants of the Rosaceae family.

In an embodiment for better results, the disease is caused by an Erwinia infection; in particular Erwinia amylovora.

In an embodiment for better results, the plant is rice.

In an embodiment for better results, the disease is a rice blast.

In an embodiment for better results, the disease is caused by a M. orzyae infection.

Another aspect of the present disclosure relates to a composition for plant protection comprising an effective amount of the Bacillus velezensis described in the present disclosure, and a suitable additive.

In an embodiment for better results, the suitable additive is selected from a list consisting of: a carrier, a solvent, a buffer, an oil, a stabiliser, a preservative, an excipient, a surfactant, an emulsifier, an adhesion agent, an emulsifier, osmoprotectant, sun protectant, wetting agent, or mixtures thereof.

In an embodiment for better results, the carrier is alginate, chitosan, ulvan, fucoidan, carrageenan, xanthan gum, gellan gum, gelatin, arabic gum, lignin, maltodextrin, zein, protamine sulphate, lecithin, gelatin matrices, methacrylate-modified lignin, methacrylated gellan gum, polyvinyl alcohol, low density polyethylene, poly(ε-caprolactone), polyethylene glycol or poly(lactic-co-glycolic acid), or combinations thereof; preferably alginate.

In an embodiment for better results, the composition may further comprise an agriculturally acceptable compound; preferably agriculturally acceptable compound is selected from: plant strengtheners, growth promoters, nutrients, fertilisers, vitamins, minerals; or mixtures thereof.

In an embodiment for better results, the composition may further comprise a biological plant protector agent; such as an essential oil.

In an embodiment for better results, the composition comprises an effective amount of B. velezensis strain in the composition in the composition ranges from 1.0×10³ CFU/mL to 1.0×10¹¹ CFU/mL, preferably from 1.0×10⁵ CFU/mL to 1.0×10¹⁰ CFU/mL; more preferably from 1.0×10⁶ CFU/mL to 1.0×10⁹ CFU/mL.

In an embodiment for better results, the composition is in the form of wettable powders, granules, flowables, microencapsulations or nanoencapsulations.

Another aspect of the present disclosure relates to a composition comprising a mixture of proteins in an effective amount, wherein the proteins are 95% identical to the following list: SEQ ID No. 1, SEQ ID No. 2, SEQ ID No. 3, SEQ ID No. 4, SEQ ID No. 5, SEQ ID No. 6, SEQ ID No. 7, SEQ ID No. 8, SEQ ID No. 9, SEQ ID No. 10, SEQ ID No. 11, SEQ ID No. 12, SEQ ID No. 13, SEQ ID No. 14, SEQ ID No. 15, SEQ ID No. 16, SEQ ID No. 17, SEQ ID No. 18, SEQ ID No. 19, and SEQ ID No. 20; preferably with a suitable additive, the additive may be any of the described additives

In an embodiment the composition comprises one protein, two proteins or three proteins, wherein the protein is 95% identical to a protein of following list: SEQ ID No. 1, SEQ ID No. 2, SEQ ID No. 3, SEQ ID No. 4, SEQ ID No. 5, SEQ ID No. 6, SEQ ID No. 7, SEQ ID No. 8, SEQ ID No. 9, SEQ ID No. 10, SEQ ID No. 11, SEQ ID No. 12, SEQ ID No. 13, SEQ ID No. 14, SEQ ID No. 15, SEQ ID No. 16, SEQ ID No. 17, SEQ ID No. 18, SEQ ID No. 19, or SEQ ID No. 20.

In an embodiment for better results, the mixtures of proteins consists in a mixture 95% identical to the following list: SEQ ID No. 1, SEQ ID No. 2, SEQ ID No. 3, SEQ ID No. 4, SEQ ID No. 5, SEQ ID No. 6, SEQ ID No. 7, SEQ ID No. 8, SEQ ID No. 9, SEQ ID No. 10, SEQ ID No. 11, SEQ ID No. 12, SEQ ID No. 13, SEQ ID No. 14, SEQ ID No. 15, SEQ ID No. 16, SEQ ID No. 17, SEQ ID No. 18, SEQ ID No. 19, and SEQ ID No. 20.

Another aspect of the present disclosure relates to the Bacillus velezensis strain described in the present disclosure or to the composition described in the present disclosure as a biological plant protection agent.

A method for protecting or treating plants and/or fruit from fungal or bacterial infections comprising applying an effective amount of the Bacillus velezensis strain or the composition described in the present disclosure.

### BRIEF DESCRIPTION OF THE DRAWINGS

The following figures provide preferred embodiments for illustrating the disclosure and should not be seen as limiting the scope of invention.

FIG. 1: Embodiment of the phylogenomic tree of B. velezensis, highlighting the placement of strain I21.

FIG. 2: Embodiment of the in vitro inhibition of E. amylovora (CM1) growth by the disclosed I21 B. velezensis by dual-plate solid and liquid assays. A: Inhibition halo of E. amylovora (CM1) growth when grown together with the Commercial (I16) strain (left panel) and I21 B. velezensis (right panel) in solid Luria-Bertani (LB) media; B: Quantification of data presented in panel A (n=3, with 3 replica per experiment); C: Inhibition of E. amylovora (CM1-GFP) when grown together with different I21 B. velezensis OD₆₀₀ as measured by fluorescence emission in liquid LB media.

FIG. 3: Embodiment of the dual-plate inhibition assay of two E. amylovora reference strains with Commercial (I16) and I21. A: Inhibition of E. amylovora CFBP 1430 by Commercial I16 (Serenade ASO; left panel) and I21 (right panel); B: Inhibition of E. amylovora Ea273 (ATCC 49946) by Commercial I16 (Serenade ASO; left panel) and I21 (right panel).

FIG. 4: Embodiment of an antibiogram of E. amylovora strains with Streptomycin. A: E. amylovora CFBP 1430; B: E. amylovora Ea273 (ATCC 49946); C: E. amylovora CM1; D: Schematic representation of the antibiogram layout (streptomycin concentrations from 0 to 2500 μg/ml on antibiotic discs in LB media).

FIG. 5: Embodiment of indole-acetic-acid (IAA) production by I21 B. velezensis strain and Commercial I16 (Serenade ASO).

FIG. 6: Embodiment of 1-Aminocyclopropane-1-carboxylic acid (ACC) deaminase activity of I21 B. velezensis strain and Commercial I16 (Serenade ASO).

FIG. 7: Embodiment of catalase activity of B. velezensis strain I21 and Commercial I16 (Serenade ASO).

FIG. 8: Embodiment of the effect of I21 treatment in reducing the infection symptoms caused by E. amylovora CM1-GFP in detached pears from the Rocha variety; A: Phenotype in intact control pears treated with PBS and infected with CM1-GFP 24 hours later; A′: Phenotype in cut pears treated as in Panel A; A″: Spread of E. amylovora CM1-GFP fluorescence in pears as in Panel A; B: Phenotype of pears treated with I21 and infected with CM1-GFP 24 hours later; B′: Phenotype in cut pears treated as in Panel B; B″: Spread of E. amylovora CM1-GFP fluorescence in pears treated as in Panel B; C: Phenotype of pears treated Commercial I16 (Serenade ASO, Bayer) and infected with CM1-GFP 24 hours later; C′: Phenotype in cut pears treated as in Panel C; C′: Spread of E. amylovora CM1-GFP fluorescence in pears treated as in Panel C.

FIG. 9: Embodiment of the effect of I21 treatment in reducing the infection symptoms caused by E. amylovora CM1-GFP in detached pears from the Rocha variety. A: Phenotype in intact control pears treated with PBS and infected with CM1-GFP 24 hours later; A′: Phenotype in cut pears treated as in Panel A; A″: Spread of E. amylovora CM1-GFP fluorescence in pears as in Panel A; B: Phenotype of pears treated with I21 and infected with CM1-GFP 24 hours later; B′: Phenotype in cut pears treated as in Panel B; B″: Spread of E. amylovora CM1-GFP fluorescence in pears treated as in Panel B. C: Quantification of I21 effect on fire blight disease progression by means of reduction of CM1-GFP expression. Percentage of inhibition was calculated as follows: 100−[(100×Mean fluorescence of Panel B″)/Mean fluorescence of A″]. Mean fluorescence as measured with ImageJ. Each experiment was performed 4 times with 6 immature pears each. The analysis was performed on 3 images per pear corresponding to each inoculation whole (6 pears×3 wholes=24 images/per treatment/experiment).

FIG. 10A: Embodiment of in vitro I21 B. velezensis efficacy studies at optical density (OD)=0.6. From left to right: Naked I21-I21 B. velezensis only; Formulation without I21-control sample of alginate containing solution; Formulated I21-I21 strain mixed in an alginate-containing solution; Empty MPs-empty alginate microparticles, used as control; I21 loaded MPs-alginate microparticles loaded with I21 B. velezensis.

FIG. 10B: Embodiment of in vitro I21 B. velezensis efficacy studies at OD=1.0. From left to right: Naked I21-I21 B. velezensis only; Formulated 121.

FIG. 11: Embodiment of viability of naked and formulated I21 B. velezensis over time.

FIG. 12: Embodiment of the effect of I21-loaded microparticles treatment in reducing the infection symptoms caused by E. amylovora CM1-GFP in detached pears from the Rocha variety. A: Phenotype in intact control pears treated with empty microcapsules and infected with CM1-GFP 24 hours later; A′: Phenotype in cut pears treated as in Panel A; A″: Spread of E. amylovora CM1-GFP fluorescence in pears treated as in Panel A; B: Phenotype of pears treated with I21 loaded microparticles and infected with CM1-GFP 24 hours later; B′: Phenotype in cut pears treated as in Panel B; B″: Spread of E. amylovora CM1-GFP fluorescence in pears treated as in Panel B. C: Quantification of the I21-loaded microparticles effect on fire blight disease progression by means of reduction of CM1-GFP expression. Percentage of inhibition was calculated as follows: 100−[(100×Mean fluorescence of Panel B″)/Mean fluorescence of A″]. Mean fluorescence as measured with ImageJ. Each experiment was performed 2 times with 6 immature pears each. The analysis was performed on 3 images per pear corresponding to each inoculation whole (6 pears×3 wholes=24 images/per treatment/experiment).

FIG. 13: Embodiment of the effect of Formulated I21 treatment in reducing the infection symptoms caused by E. amylovora CM1-GFP in detached pears from the Rocha variety. A: Phenotype in intact control pears treated with formulation mixture control and infected with CM1-GFP 24 hours later; A′: Phenotype in cut pears treated as in Panel A; A″: Spread of E. amylovora CM1-GFP fluorescence in pears treated as in Panel A; B: Phenotype of pears treated with formulated I21 and infected with CM1-GFP 24 hours later; B′: Phenotype in cut pears treated as in Panel B; B″: Spread of E. amylovora CM1-GFP fluorescence in pears treated as in Panel B; C: Quantification of the formulated I21 effect on fire blight disease progression by means of reduction of CM1-GFP expression. Percentage of inhibition was calculated as follows: 100−[(100×Mean fluorescence of Panel B″)/Mean fluorescence of A″]. Mean fluorescence as measured with ImageJ. Each experiment was performed 2 times with 6 immature pears each. The analysis was performed on 3 images per pear corresponding to each inoculation whole (6 pears×3 wholes=24 images/per treatment/experiment).

FIG. 14: Embodiment of the effect on cell viability after spray-drying of I21 with magnesium sulphate.

FIG. 15: Embodiment of A) Macroscopic view of in vitro antifungal activity of naked and formulated I21 against M. oryzae at 7-days post-incubation. B) Inhibitory effects of naked and formulated I21 on mycelial growth of M. oryzae at 4 and 7-days. Data are presented as mean±standard deviation (N=3). Data were analyzed by one-way analysis of variance (ANOVA), followed by Tukey's multiple comparison post hoc test (*p<0.05 **p<0.01, ***p<0.001 vs No treatment). The normal distribution of the data was assessed by the Shapiro-Wilk test. Statistical analysis was performed using Prism GraphPad 8.02 Software® (GraphPad Software, La Jolla, CA, USA). ‘MPs’ signifies microparticles.

FIG. 16: Embodiment of in vitro growth inhibition of the virulent M. oryzae Portuguese isolate PR9GFP by decreasing concentrations of B. velezensis I21 in dual-liquid assays measured by means of fluorescence in a plate reader (BioTek Synergy H1 Plate Reader).

FIG. 17: Embodiment of the effect of I21 treatment in reducing the infection symptoms caused by the virulent M. oryzae isolate M22.7 in detached rice leaves from the rice blast-susceptible Presto cultivar. A) Phenotype in detached rice leaves treated with PBS (control) and infected with M22.7 24 h later; A′) Detail of a rice leaf with rice blast lesions from Panel A; B) Phenotype in detached rice leaves treated with I21 and infected with M22.7 24 h later; B′) Detail of a rice leaf from Panel B. Each experiment was performed 3 times, with 3 plates containing 5 leaves each.

### DETAILED DESCRIPTION

The present disclosure relates to a Bacillus velezensis strain, and a composition for plant protection comprising an effective amount of said Bacillus velezensis. The use of the disclosed Bacillus velezensis strain or composition as a biological plant protection agent, and a method for protecting or treating plants and/or fruit from fungal or bacterial infections comprising applying an effective amount of the Bacillus velezensis strain or the composition, are also disclosed.

In an embodiment, the B. velezensis strain of the present disclosure can be used as a BPPA against phytopathogenic Erwinia bacteria in plants, in particular plants of the Rosaceae family. In a further embodiment, the B. velezensis strain of the present disclosure can be used as a BPPA to prevent or suppress fire blight caused by E. amylovora infection.

In another embodiment, the B. velezensis strain can be used as a BPPA to prevent or suppress rice blast caused by M. orzyae infection.

In an embodiment, the BPPA may exert its effect against the pathogens through several mechanisms, including, but not limited to, occupation of growth space otherwise free to the pathogen, creation of micro-environments and biofilms that disfavour colonisation, or release of biosynthetic metabolites/peptides with antimicrobial properties that interfere with pathogen biology.

In an embodiment, the composition comprising the I21 B. velezensis strain may contain a solvent, water and/or an acceptable buffer.

In a preferred embodiment, the composition comprising the I21 B. velezensis strain may contain other elements which serve to stabilise the active ingredient and allow application in the field using contemporary agricultural equipment, protect the bacteria from premature degradation due to humidity, temperature and solar ultraviolet light exposure, maintain the viability of the active ingredient over time and finally control the release rate of the active ingredients over time. Agriculturally acceptable compounds may be added to the composition comprising the I21 B. velezensis strain in order to extend shelf-life and optimise the processability and physicochemical characteristics of the formulation. Such agriculturally acceptable compounds can include, but are not restricted to, plant strengtheners, growth promoters, nutrients, wetting agents, solvents, buffers, stabilisers, osmoprotectants, sun protectants, excipients, surfactants, adhesion agents, fertilisers, emulsifiers, vitamins, minerals, additives and/or preservatives and so on.

In a further embodiment, in order to improve efficacy, onset and duration of action, the composition comprising the I21 B. velezensis strain may be augmented with at least one other BPPA, that can comprise, but not restricted to, essential oils, other beneficial microorganisms, extracts from microorganisms or other natural, semi-synthetic or synthetic biodegradable and non-toxic biomolecules.

In another aspect, the composition comprising the I21 B. velezensis strain may be in the form of a liquid, concentrate, tablet, gel, pellet, wettable powder, granule or flowable, amongst others. In a preferred embodiment, the formulation is in the form of microcapsules (size range 1-999 μm) or nanocapsules (size range 1-999 nm) wherein the active ingredient is initially retained within the interior of the capsule shell. The microcapsules or nanocapsules may be prepared from one or more materials that may be selected from natural-origin polymers, proteins or lipids, such as, but not limited to, chitosan, alginate, ulvan, fucoidan, carrageenan, xanthan gum, gellan gum, gelatin, arabic gum, lignin, maltodextrin, zein, protamine sulphate or lecithin.

In another embodiment, the microcapsules or nanocapsules comprising the I21 B. velezensis strain may be prepared from one or more non-toxic, biodegradable synthetic or semi-synthetic materials such as, but not limited to, methacrylate-modified lignin, methacrylated gellan gum, polyvinyl alcohol, low density polyethylene, poly(ε-caprolactone), polyethylene glycol or poly(lactic-co-glycolic acid).

Through judicious choice of the encapsulation material(s), variation of the polymer concentration, crosslinking density, interior pore size, capsule size, shape and surface charge, it is possible to tune the loading capacity, entrapment efficiency and release kinetics of the entrapped active ingredient. Suitable techniques for microencapsulation include ionic gelation, spray-drying and microfluidics, the latter being suitable also for nanoencapsulation. By optimisation of parameters such as air temperature, feed flow rates and so on, it is possible to fine-tune the physicochemical properties of the capsules, which can, if necessary, be further processed downstream by techniques such as ball-milling and drying by conventional means or on a fluid bed dryer.

In a preferred embodiment, the concentration of the I21 B. velezensis strain in the composition of the present disclosure is in an effective range, preferably between 1.0×10³ CFU/mL and 1.0×10¹¹ CFU/mL, more preferably between 1.0×10⁵ CFU/mL and 1.0×10¹⁰ CFU/mL and even more preferably between 1.0×10⁶ CFU/mL and 1.0×10⁹ CFU/mL. Preferably, the composition comprises 0.1-5% (w/w) of the disclosed I21 B. velezensis strain, more preferably between 0.5-4% (w/w), and even more preferably between 1-3% (w/w).

In an embodiment, the plant or fruit to be treated is selected from the Rosaceae family, preferably but not limited to, an apple or a pear plant. The I21 B. velezensis composition may be applied in sufficient amounts to thoroughly wet any of the above ground structures of the plant, including leaves, flowers, stems, trunk, bark, blossoms, buds and fruit, without significant run-off of the applied material. In another embodiment, the plant is a rice plant.

Another embodiment comprises treatment of the plant before infection (prophylactic application) or after detection of infestation by applying posteriorly the composition comprising the disclosed I21 B. velezensis to minimise the damaging effects caused by the pathogen. At least one further application of the composition may be applied to adequately control the pathogen. This embodiment also provides a formulation for control or suppression of the pathogen that extends the duration of effectiveness of the disclosed composition.

In an embodiment, application of the composition comprising the I21 B. velezensis is not dependent upon whether the plants have received prior treatment with other plant protection agents of whatever class.

In an embodiment, the composition comprising the I21 B. velezensis may be applied at any time. In a preferred embodiment however, at least a first application to the plant takes place at the early flower stage, for example from appearance of the first stamen to full bloom.

### Examples

1. Isolation of E. amylovora (Burrill) Winslow et al. (CM1) from Pyrus communis Cultivar Rocha.

In an embodiment, secondary shoots from a fire blight symptomatic pear orchard (Pyrus communis cultivar Rocha) were collected from the Campo Maior region in Portugal. The plant stems were de-barked, cross-sectioned, disinfected with 70% (v/v) ethanol and stamped onto Kings B (KB) agar media (20 g/L proteose peptone, 10 mL/L glycerol, 1.5 g/L K₂HPO₄, 1.5 g/L MgSO₄·7H₂O, 15 g/L agar). Morphologically different bacteria were selected and re-streaked several times onto KB to obtain single colonies. To molecularly identify the bacteria, genomic DNA was extracted using a quick DNA extraction protocol in which individual bacterial colonies were re-suspended in 500 μL of 0.9% (w/v) NaCl, followed by centrifugation at 12,000×g for 3 min at room temperature. The supernatant was discarded, the pellet re-suspended in 100 μL NaOH solution (20 mM) and vortexed for 1 min in a turbo vortex (this step was repeated twice). The extraction solution was incubated at 100° C. for 5 min, with two cycles of turbo vortexing for 2 min, and then sonication on a water bath for 1 min. To identify E. amylovora amongst the isolated bacteria, a PCR was performed with the primers pEA71Fw and pEA71Rev (pEA71Fw (SEQ ID No. 69, Taylor et al, 2001)-CCTGCATAAATCACCGCTGACAGCTCAATG; pEA71Rev (SEQ ID No. 70, Taylor et al., 2001)-GCTACCACTGATCGCTCGAATCAAATCGGC, respectively) that amplify pEA71, a genomic region specific of this strain (Taylor, 2001). The PCR amplification step was as follows: 3 min of denaturation at 94° C., followed by 35 cycles of 30 sec of denaturation at 94° C., 20 sec annealing at 58° C. and 12 sec of extension at 72° C. finishing with an extension step of 5 min at 72° C. One colony produced the expected PCR fragment of 187 bp and was identified as E. amylovora and named CM1. The molecular identification was further confirmed by amplification and sequencing a partial sequence of the 16S rRNA gene. The PCR amplification step was as follows: 3 min of denaturation at 94° C., followed by 35 cycles (each 30 sec) of denaturation at 94° C., 20 sec annealing at 55° C. and 1:10 min of extension at 72° C., finishing with an extension step of 5 min at 72° C. A 1% (v/v) agarose electrophoresis was performed to confirm amplification of the PCR product. PCR products were purified with a NZYGelpure kit (NZYTech, Lisbon) and Sanger sequenced with the same primers used for amplification (StabVida, Caparica). The sequences were manually trimmed, searched against publicly available databases and confirmed the identification as E. amylovora. Stocks for long term storage were made for all isolates in a final volume of 25% (v/v) glycerol and kept at −80° C.

For the scope and interpretation of the present disclosure it is defined that “room temperature” should be regarded as a temperature between 15-30° C., preferably between 18-25° C., more preferably between 20-22° C.

Methods for the alignment of sequences for comparison are well known in the art, such methods include GAP, BESTFIT, BLAST, FASTA and TFASTA. GAP uses an algorithm (Needleman, 1970)) to find the global (over the whole the sequence) alignment of two sequences that maximizes the number of matches and minimizes the number of gaps. The BLAST algorithm (Altschul, 1990)) calculates percent sequence identity and performs a statistical analysis of the similarity between the two sequences. The software for performing BLAST analysis is publicly available through the National Centre for Biotechnology Information (NCBI). Global percentages of similarity and identity may also be determined using one of the methods available in the MatGAT software package (Campanella, 2003). MatGAT: an application that generates similarity/identity matrices using protein or DNA sequences). Minor manual editing may be performed to optimise alignment between conserved motifs, as would be apparent to a person skilled in the art. The sequence identity values, which are indicated in the present subject matter as a percentage were determined over the entire amino acid sequence, using BLAST with the default parameters.

2. Isolation of B. velezensis Strain I21 from Pyrus communis Cultivar Rocha.

Secondary shoots from a fire blight symptomatic pear orchard (Pyrus communis cultivar Rocha) were collected from the Oeste region in Portugal. The plant stems were treated as above and stamped onto Nutrient Agar (NA, Biolife) and KB agar media. Morphologically different bacteria were selected and re-streaked several times to obtain single colonies. Long term storage and genomic DNA (gDNA) extraction were performed as above. To molecularly identify the bacterial species isolated, a partial sequence of the 16S rRNA gene (1.5 Kb length) was amplified and sequenced. For that, a volume of 1 μL of gDNA was used for PCR. The PCR amplification step was as follows: 3 min of denaturation at 94° C., followed by 35 cycles (each 30 sec) of denaturation at 94° C., 20 sec annealing at 55° C. and 1:10 min of extension at 72° C., finishing with an extension step of 5 min at 72° C. A 1% (v/v) agarose electrophoresis was performed to confirm amplification of the PCR product. PCR products were purified with a NZYGelpure kit (NZYTech, Lisbon) and Sanger sequenced with the same primers used for amplification (StabVida, Caparica). The sequences were manually trimmed, searched against publicly available databases and identified as Bacillus sp. To further characterise the strain, two Bacillus genes rpoB (SEQ ID No 28) (Rooney, 2009) and gyrA (SEQ ID No 29) (Rooney, 2009) were amplified and sequenced. The database search and phylogenetic analysis revealed that the strain belongs to the genus Bacillus velezensis, having high sequence similarity to strain ZeaDK315Endobac16, with the GenBank accession number CP043809.1.

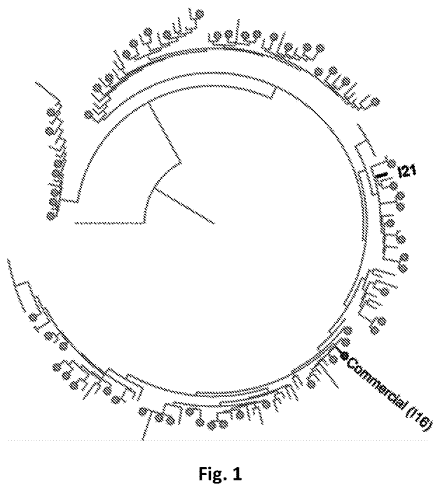

In an embodiment, to obtain a deeper molecular characterization of the Bacillus velezensis I21 strain, whole-genome sequencing was carried out. Genomic DNA was extracted following a modified version of the Marmur protocol (Salvà-Serra 2018). Library preparation was done using the Illumina Nextera XT kit, following a low-volume protocol (Baym 2015) Whole-genome sequencing was done on an Illumina NextSeq 500, which generated 2.16 million 150 bp paired-end reads. Quality control, trimming and filtering of raw reads was done with Fastp v. 0.23.2, with 2.14 million reads being retained after filtering and trimming. Genome assembly was done with SPAdes v 3.15.5 (Prjibelski 2020) with the—isolate option. Contigs with read coverage <30 (calculated using bbmap) or shorter than 500 bp were discarded. CheckM v1.2.2 (Parks 2015) was used for the assembly quality control, indicating a high-quality assembly, with high completeness (99.41%) and 0% contamination. Quast v 5.2.0 (Gurevich 2013) was used to calculate assembly statistics. This revealed a total genome size of 3,947,764 bp, across 19 contigs, with an N₅₀ of 1,002,146 bp and a GC content of 46.4%. Genome annotations were made using Prokka v 1.14.6 (Seemann 2014) and eggNOG mapper v2.1.10 (Cantalapiedra 2021). EggNOG was run with database version 5.0-2 and flags—itype genome and—prodigal. Prokka was run with—genus set to Bacillus and a protein file from the B. velezensis reference genome GCF_000769555.1 (NCBI accession), to use as priority for annotations. In total 3882 genes were identified, including 3812 coding sequences and 70 noncoding sequences. GTDB-tk v.2.2.6 (Chaumeil 2022) was used to obtain a genome based taxonomic assignment (using reference data version r207). In agreement with the target gene approach (above), the results identified the strain as B. velezensis. To better characterize the genetic relationships of the strain with other B. velezensis strains for which genomes are available, GtoTree v.1.7.08 (Lee 2019) was used to construct a phylogenomic tree (FIG. 1), using as input all genomes identified as B. velezensis in the GTDB database plus the reference genome for B. siamensis (to use as outgroup; total number of genomes in the tree=498). This allowed to show that while there are multiple clades of B. velezensis with highly similar strains (very short/absent branch lengths), the disclosed I21 strain is clearly distinct from its closest relatives. The strain is placed in a clade with 3 strains that have been isolated from South Africa (2) and France (1). One of these strains is ZeaDK315Endobac16, which was identified as the closest relative when using the target gene approach (above). The identified genome comprises the sequences SEQ ID No. 30 to SEQ ID No. 48.

FIG. 1 depicts the global phylogenomic tree of B. velezensis. As the tree includes 498 genomes, clades were collapsed at a specific branch length to enable better visualisation. Collapsed clades are indicated by points, including ≥2 closely related strains. B. velezensis I21 is shown in black, and the grey point indicates the placement of the Commercial (I16) strain, which belongs to a clade with 12 closely related strains.

In an embodiment, to understand if the I21 strain carried any proteins that distinguish it from other B. velezensis strains, pangenome analysis was carried with PanACoTA v.1.4.0 (Perrin 2021), with all the B. velezensis genomes used for the phylogenetic tree. This analysis first identifies the protein families present across all genomes (at 80% sequence identity and alignment coverage), followed by the creation of a presence absence matrix that allows the identification of proteins present in specific strains. This analysis led to the identification of 21 proteins, that are present in the I21 strain and not in any other B. velezensis strain. These protein sequences (SEQ ID No 1 to SEQ ID No. 20) were subsequently used for blast searches in order to identify their putative functions (protein sequences, their functions and respective gene sequence are provided in table 1; as 2 proteins are duplicates, only 20 sequences are provided in the table).

TABLE 1
Protein sequences present in the I21 strain and
not in any other B. velezensis strain.

| Seq. | | Organism | |
|---|---|---|---|
| ID | Sequence name | name | Residues |
| Seq. | L-dopachrome tautomerase- | B. velezensis | MKPMLPFEKYFGDLELVYAFYGAMPTGVTVSETG |
| ID. 1 | related protein (closest | | RIFICFPKWGDDVKFTVAEIIGDKLQPYPDLQTN |
| | BLAST homolog; 51% | | LVNPENIKMTFISVQSVVTDGRGTLWVLDTAAPN |
| | alignment coverage, 100% | | FSKPIKGGAKLVAVDLKTNTIRRVYTFTEDVVLP |
| | sequence identity; B. | | TTYLNDVRFDFRVGTAGYAYITDSSSKGPGAIIV |
| | velezensis) | | VDLETGNAFRRLNGEQIQLHRIPILYRK |
| Seq. | L-dopachrome tautomerase- | B. velezensis | MNRNRDGSTSPFRLASDGIAISPDGKVLFFCPLT |
| ID. 2 | related protein (closest | | SRHLFSISTEALRDRNIQDMDLLYHVAYWGEKGA |
| | BLAST homolog; 67% | | SDGMITDAKGAIYAGDYENNSIRKMLPNGTTETI |
| | alignment coverage, 96% | | AHDPRILWPDTFSIGPDGYLYVIVNQLHRQARFH |
| | sequence identity; B. | | YGKDLRQKPYSLLRIKIDEFPAPAF |
| | velezensis) | | |
| Seq. | Terminase small subunit | B. velezensis | MKLTEKQKRFADFYIELGNATEAARKAGYSQKTA |
| ID. 3 | (closest BLAST homolog; | | KSIGQENLTKPDIKSYIKERLAEKDAERVASQDE |
| | 100% alignment coverage; | | ILEFLTAVMRGEKTEQIPVGIGEGAQELEDKDPY |
| | 100% sequence identity; | | LKDRVKAAELLGKRHGMWTEKVDMTGEFAVQFID |
| | B. amyloliquefaciens) | | DSGDEE |
| Seq. | Cell wall-binding protein | B. velezensis | MKKLFKSIILSAALLTGAVAVAPSASAAWSGWQN |
| ID. 4 | YqgA precursor (closest | | ESGYSGRVFTDATTYTAGASTIDWKAEKKGDSGK |
| | BLAST homolog; 44% | | RNYIGTFESAKFQRGIRWR |
| | alignment coverage; 95% | | |
| | sequence identity; | | |
| | Streptococcus pneumoniae) | | |
| Seq. | YycA Aldo-keto reductase | B. velezensis | MAFRLFFHEENFGKTVSQLKKLVDEGKVKYFLLS |
| ID. 5 | (AKR) superfamily | | GNNSGNTKLVSWIKKNGTKNSADPYSTEKNKLSP |
| | (closest BLAST homolog; | | ARNDRRPGRTKSADSV |
| | 75% alignment coverage, | | |
| | 82% sequence identity; | | |
| | B. velezensis) | | |
| Seq. | MarR family transcriptional | B. velezensis | MVNSLKLVQRAELWDEQNENIVEKLYVDHLPNDG |
| ID. 6 | regulator (closest BLAST | | LLREIIQDDTVFLVGKKTGESTIFTRAQEYSRIS |
| | homolog; 16% alignment | | KSEISAYVDVKTMYGKSQVKELNEDSDYCKTLLC |
| | coverage, 80% sequence | | NCFIKSSNKRLIKRSE |
| | identity; B. velezensis) | | |
| Seq. | DUF4181 domain-containing | B. velezensis | MSLLPLNSALCLIEWRNLQAPHPYETSDTFLGGR |
| ID. 7 | protein (closest BLAST | | QLLIFSAVFILLSLMFQVFAVKVLNLPKPVKKQK |
| | homolog; 76% alignment | | PINGTHRLIEITLLITGIAAVNFFNEMYVFAGVV |
| | coverage, 98% sequence | | FMLFCCTGIMEWRHRREEKQYVYDSFYAGLSAAL |
| | identity; Bacillus sp.) | | GVLIFFIS |
| Seq. | Phage integrase SAM-like | B. velezensis | MHSENRKGKRVKAGRASVKTTRKGTYDLEYIFDR |
| ID. 8 | domain-containing protein | | LCDAKRAENVFRKRLISTCVLVDICRIMLKRSEC |
| | (closest BLAST homolog; 27% | | PQTYVILTLTLRAALYRG |
| | alignment coverage; 90% | | |
| | sequence identity; B. | | |
| | amyloliquefaciens) | | |
| Seq. | isocitrate/isopropylmalate | B. velezensis | MKTYKIASIPGDGVGKETVPQAERVLNAIAELHG |
| ID. 9 | family dehydrogenase | | GIAFDFK |
| | (closest BLAST homolog; 43% | | |
| | alignment coverage; 100% | | |
| | sequence identity; Bacillus | | |
| | sp.) | | |
| Seq. | hypothetical protein | B. velezensis | MLKRFFLVFLTLIMSIFAVFAAAEESPGDRVISF |
| ID. 10 | (closest BLAST homolog; 55%% | | DEPMIVLISLGIILCLFIPPFVMSFFGNLVVRII |
| | alignment coverage; 97% | | SGSLSKFYRPHVWGAHSDWISDSQWLFDNSCCRP |
| | sequence identity; Bacillus | | GNFGQFSQCCGHLIRWQDREHK |
| | sp.) | | |
| Seq. | DUF4573 containing domain | B. velezensis | MAPGSPVAPVGPVTPVIPVTPVGPVAPVEPVTPV |
| ID. 11 | (closest BLAST homolog; | | APVSPVAPVGPVIPVGPVSPVAPVTPVGPVSPVA |
| | 99%% alignment coverage; | | PVTPFAPVIPVAPVTPVAPMAPVAPVTPVEPVIP |
| | 64% sequence identity; | | VGPVAPVGPVAPVEPVVPVTPARPVSPVAPVSPV |
| | Clostridium beijerinckii) | | APVTPVGPVGPVGPVGPVIPVEPVTPVAPVGPVD |
| | | | PVIPVGPVTPVEPVSPVTPVGPVPPVAPVSPVGP |
| | | | VGPVAPVEPVGPVTPVIPVVPVVPVAPMAPVAPV |
| | | | SPVGPVSPVEPVAPVRPVSPVAPVTPVEPVEPVT |
| | | | PVAPVIPIDPVTPVTPVVPVGPVAPVEPVSPVEP |
| | | | VTPVEPVDPISPVGPVAPVGPVAPIEPVAPVGPV |
| | | | SPVGPISPVTPVAPVSPVEPVEPVAPVIPVDPVT |
| | | | PVPPVSPVTPVEPVGPVGPVEPVAPVTPVAPVIP |
| | | | VAPVTPVGPVTPVGPVAPVDPVEPVIPVAPVGPI |
| | | | SPVTPVAPVSPIEPVEPVGPFSPVAPVTPVAPVS |
| | | | PVGPVSPVTPVTPVEPVVPVTPVIPVGPASPVAP |
| | | | VSPVEPVSPVAPVTPVAPVTPVEPVAPVSPVEPV |
| | | | TPVDPVGPVAPVIPVEPVDPVGPFSPVGPISPVT |
| | | | PVAPVGPVSPVGPISPVTPVAPVGPVSPVTPVTP |
| | | | VAPVTPVAPVTPVEPVVPVEPVSPVAPVGPVAPV |
| | | | TPVAPVTPVEPVVPVSPVGPVTPVSPVEPVVPIG |
| | | | PVSPVIPVAPIGPVSPVIPVAPVGPVSPVIPVAP |
| | | | VGPVSPVIPVAPVGPVVPVPPVTPVAPVGPVDPV |
| | | | APVTPVEPVAPVDPVTPVAPVDPVTPVAPVSPVE |
| | | | PVTPVAPVSPVEPVIPVAPVAPVVPVEPVEPVSP |
| | | | VAPVAPVTPVAPVSPVEPVIPVAPLSPVEPVTPV |
| | | | EPVAPVVPVTPVGPVSPVGPVSPVIPVAPDAPVA |
| | | | PVSPVDPVSPVDPVAPVTPVEPVAPVTPVGPVTP |
| | | | VDPVSPVGPVSPVIPVGPAAPVGPVTPVEPVDPV |
| | | | TPVGPVDPVDPVAPVGPIAPVAPVDPVDPIAPVT |
| | | | PVGPVSPVAPVTPVAPVDPVTPVGPVVPVAPVIP |
| | | | VAPVAPDAPVDPVSPVDPVTPVGPVEPVDPVAPV |
| | | | GPAAPVVPVAPVEPVAPIEPVNPVGPVTPAGPVA |
| | | | PVEPVGPISPVEPVAPVTPVNPVGPVSPVAPVEP |
| | | | VTPVVPVTPVAPVSPVGPVIPVDPVAPDAPVTPV |
| | | | GPAAPVEPVSPVAPVTPVTPVDPMTPVGPVTPVD |
| | | | PVNPVSPVEPVTPVVPVTPVAPVSPVGPVAPVGP |
| | | | VTPVAPVSPVTPVTPVAPVIPVAPVSPVEPVAPV |
| | | | GPVTPVAPIGPVTPVGPVAPVEPVEPVAPVSPVD |
| | | | PVGPVSPVEPVDPVSPVDPVTPVDPVTPVGPVTP |
| | | | VSPVDPVDPVAPVTPVGPVSPVGPVTPVAPVSPV |
| | | | GPVIPVAPVGPMGPVAPVAPVIPVAPVTPVDPVS |
| | | | PVDPVAPVTPVEPVTPVTPVEPVAPVDPVAPVEP |
| | | | VAPVTPVEPVEPVAPVTPVGPVTPVAPVSPVGPV |
| | | | APVAPVTPVEPVDPVKPVDPVKPVDPVKPVGPVA |
| | | | PVSPVGPVAPVEPVVPVPPVTPVTPVDPVDPVAP |
| | | | VSPVGPFSPVGPVEPVTPVAPVGPVGPVIPVAPV |
| | | | TPVAPISPVAPVTPVAPVDPVAPVEPVAPVGPIG |
| | | | PVTPVAPVIPVAPVTPVDPVTPVGPVSPVEPVAP |
| | | | VVPVIPVGPVTPVGPISPIGPVSPVEPVEPVDPV |
| | | | APVGPVLPAEPVTPVDPVTPVGPVEPVGPVAPVE |
| | | | PVDPVTPVGPVTPVAPVMPVAPVAPVDPVAPIEP |
| | | | VNPVGPVTPSWPVTPVAPVTPVDPVTPVGPVTPV |
| | | | GPDAPVDPVTPVAPVVPVEPVVPVAPVEPVPPVT |
| | | | PLGPVTPVGPVTPVGPVIPVTPGSPPVCANETSS |
| | | | IKIVPVETLLNTMTAEAAPEGTLLVL |
| Seq. | restriction endonuclease | B. velezensis | MLNDYIKPSILWTDVEDRIDATFYRTDFVVSLDK |
| ID. 12 | subunit S (closest BLAST | | IFKNLTIPVLPNRQLIHELTDYTANGSFESLKKN |
| | homolog; 43% alignment | | VRYTNEAGIPMIRGKNLKNGVLDKTDLNYVTDDT |
| | coverage; 45% sequence | | YRFLKKSELNAGDLLFTKTGTLGNTIVWSEYFGK |
| | identity; Paraclostridium | | ATLGDNIFKVRYKNGINPYYIHAYFSTDYGYKWV |
| | bifermentans) | | ERFAQGGVQPTIILALVKSEISAEPLVIPLPNTS |
| | | | DPL |
| Seq. | N-6 DNA methylase (closest | B. velezensis | MQALIEELKEQLMMKGYSTFSGPETHEEDGTTIF |
| ID. 13 | BLAST homolog; 100% | | LASGDEKIVYVKVFDKGADLSFEVQDAALIEAQY |
| | alignment coverage; 95% | | HEEAPDFIWASNGEVNYYADCIESDPLAEIPAVI |
| | sequence identity; B. | | DVTKKEKRQISKQEEWSRKMYSTLQKRFDDLHER |
| | subtilis) | | LYGPGGADNISSTNEAIDELSKLLFMETFRLYHP |
| | | | DYVLREGENAGLRILDILKYQYIKENGKEAVRQI |
| | | | RDAFKEIKNHEDYVAVNDLGEECFIFSEDEYIRL |
| | | | ENPANFIAAFETFQELDDFEDENGNRVKATLKNV |
| | | | SADILGRLFDVLMRGKYDNKIGQATYLTPRQVTE |
| | | | AMIEMVVHDLTSNPKEASKLITADEKGIPTFRMG |
| | | | DITCGSGGFLIKGYEAVKKYMLTQFTGTMKEKME |
| | | | KHFELMKEHSFVGADKTRNMVLKARLNMAMHGLP |
| | | | KAPIFWVNDSLMTESLKPKSFDLIVTNPPFKSGS |
| | | | VSSKTDEGKAILERYSEGIDETGKPKKEGLCLGA |
| | | | KPNPKGIWKEVSSTDQAVLFIDRNLQLLKDGGQL |
| | | | IIVLPDGILSNSGYKHVREYLMGKKNEVTGEFEG |
| | | | GKGIVKAVVSLPTVTFQLSGTGAKTSFLYIKKKE |
| | | | HENEKQGPVFMAVAEEIGFDVKNKIEVQLGNDRN |
| | | | DLLKIVDVYKKGVN |
| Seq. | hypothetical protein | B. velezensis | MRLILHEYLTIPKTTEQLVNIAKEHGLNWNEEQV |
| ID. 14 | (closest BLAST homolog; | | KLYCLLDPSIYETESGQWAVKVDDRRHTILGAIE |
| | 100% alignment coverage; | | KALEKRPMTKIDPNIIEQLPSDMIVPHNEVIEVA |
| | 98% sequence identity; B. | | VSTGRYESPRENIIRVKRK |
| | subtilis) | | |
| Seq. | class III lanthipeptide | B. velezensis | MKEVLVLQQFDEKSEVEPTAWTVTLTTISVTLST |
| ID. 15 | (closest BLAST homolog; | | LSNHC |
| | 100% alignment coverage; | | |
| | 100% sequence identity; | | |
| | B. amyloliquefaciens) | | |
| Seq. | class III lanthionine | B. velezensis | MDKKLVYFQHMSEDSKYYIEKSPNAETDPYEVHN |
| ID. 16 | synthetase LanKC | | IPNHYTVSTEYKSPWKFYNNPNHNLKEQGWKIHV |
| | (closest BLAST homolog; | | SATMNNAQDILKVVSQIMFKHQIPFKHIKDKQSL |
| | 100% alignment coverage; | | FNMNSKHGNRTSSGKFIAIYPSSNNMFFDLLNEL |
| | 97% sequence identity; | | YEELKHYENGPYILNDKSWKDSNIYYRYGAFKNI |
| | Bacillus sp.) | | YNERGELCIKDENGNLIPDNRNPYYKIPDFVSDF |
| | | | DQFLDTKNNLLDTQDVSNFNDYEFQGVLRFNNGG |
| | | | GIYTGIRKSDGKKVVIKEGRPKVGLDGQYNDAID |
| | | | RLNIEYEALNKLTEVEGVVKALDYFKAWKHVFLV |
| | | | EEFVEGVTLQSWCAINYPFNVETDKNEYISKVKK |
| | | | IINSLIKAIKLMHSKNVGMGDLQPQNIMITPELN |
| | | | VKIIDFEAASALSEENGALQTIGYTHKKNKNHKE |
| | | | RDWYALKKVLRFCILPIGPVVDIEETIKKKHDMW |
| | | | IEEEFGKDVYQYVVNTENLCDKYLSDTKEKRFNN |
| | | | NYDSKIQKEMDMSKIIKGLRKNIESNILPNSSLI |
| | | | HGDVRQFVMPSGKLNVLTGGTGAALALYRSGNIN |
| | | | KRVQDWIEIVLLENLNVLNDDIGLFTGKAGIAVT |
| | | | LYELGYTEQAVDIFNNLSYEDMKDISLRSGLSGI |
| | | | GLSLISLYLEENNDDLLVTSEKIADQIENRIIED |
| | | | DELSVTDFAAVPIGLIDGWSGISLFYAALYSVTN |
| | | | NEKYYLKSKEYIERDLKHCKVDEETNTLQTYDDQ |
| | | | NPRLLPYLSGGSIGIGIAIWYLNYVRDTNLFREE |
| | | | LEQIINLNKVRIAYSGGLFDGIGSFLLIPPMTKE |
| | | | KNEISQHIELAMKKLELFLIRKDDQILFPGNFSF |
| | | | RLADDYYSGSAGIILGINSIQFSNPLYWLPIIKI |
| | | | DSFLRKTKKELLLNN |
| Seq. | ABC transporter ATP-binding | B. velezensis | MMTSPKFKSIEFFFLLRKTRPNIRLFILAIFLSG |
| ID. 17 | protein (closest BLAST | | LSAIASTLVPHYLKSLIDNYNILDKINLRDLLGL |
| | homolog; 100% alignment | | LGIFLSSGIIGAVGSYMLSVVGYNVVANLRNLLW |
| | coverage; 96% sequence | | EKIVILPVEFYDKNRSADIASRFVNDTTVIYNLV |
| | identity; B. vallismortis) | | SNSFSQFINAILIVLFCGFWLFYYDWQLTLVIIV |
| | | | AIPIYLLIFIPLGRILSKLSKMTQSLTGKLNVRA |
| | | | VETISSIRLIKSSTAEKLQVEKGRDDIEALRNIG |
| | | | LRQTKWMVILNPIINLMMMLIIISIIGFGGVKLA |
| | | | NGSLSPGTFIAFLTLMFYIIGPLNNLGLFFTQIQ |
| | | | KTIGATERISQLIYEQGEDQHKGKELAETYKDLV |
| | | | IENLSFCYGKEDKAFSLNNINLVIKRGSVNALVG |
| | | | PSGSGKTTIISILERYYKPNSGSIYFGEENIELY |
| | | | SLKSWRSQIGYVAQNHDLISGTVRDNLIFGLENT |
| | | | PNENRIVEACKKAYAWQFISELPNSLETNIGEKG |
| | | | LNLSGGQRQRIAIARMFLKNPNIIFLDEATASLD |
| | | | SHSEQIVNEAMKKLMKDRTAIIIAHRLSTIINAD |
| | | | NIIFMENGEVTGQGNHWELKRSHKLYHQFCEQQF |
| | | | KEDSTDPLVLTN |
| Seq. | S8 family serine peptidase | B. velezensis | MKNLRRISMVFILLFVSSLQPISSHAEESNYDES |
| ID. 18 | (closest BLAST homolog; | | IIINLDSKVSESTINKVKDRYSNIGTNYIKELDL |
| | 100% alignment coverage; | | LVIDNIKPSQFSAIKDIAKEKLDAKEIIEDKEIS |
| | 94% sequence identity; B. | | LPSKQLSSKLAISSQFTEDNDTLYDSWLWNIKQV |
| | halotolerans) | | TNDYRSYRINKGSHNTKVGVIDSGIDFNHPDLKN |
| | | | NIVDTGESFVPVEDNTQDFYGHGTSVAGIIAANG |
| | | | DVKGVGPNLGLVPYKVFDKNGKSKTSWVISAIVK |
| | | | ATEDDMDVINLSLSTFLSKKDKEDRETIKLFEKA |
| | | | FKFARKHNTLIVAAAGNHGYDISNSKKLAQQLGK |
| | | | PKDNILLLPGGDNDVITVSATTAQNTKASYSNYG |
| | | | KTISIAAPGGDLDPSFLDTNKVDLYSLVWTTSPV |
| | | | NIPQTPLSKSLGFQKGYEPTLGTSVAAPIVTATA |
| | | | ALLKTEYYEKYGVEMSNRKVEKNLYQNTDHFENL |
| | | | NKNEVGSGIVNAYESLLNIER |
| Seq. | class III lanthipeptide | B. velezensis | MNNVLALQQLNKSAEVEPQVGPTITTITVIPTMV |
| ID. 19 | (closest BLAST homolog; | | ISLISNYCDNHAS |
| | 100% alignment coverage; | | |
| | 100% sequence identity; | | |
| | B. nakamurai) | | |
| Seq. | class III lanthipeptide | B. velezensis | MRDVLELQQLNEGTEVEASKGKGGLLTLITTVVG |
| ID. 20 | (closest BLAST homolog; | | TSTISNNCG |
| | 100% alignment coverage; | | |
| | 100% sequence identity; | | |
| | B. subtilis) | | |
| Seq. | L-dopachrome tautomerase- | B. velezensis | ATGAAACCTATGCTGCCTTTTGAAAAATATTTTG |
| ID. 49 | related protein - gene | | GGGATTTAGAACTGGTTTATGCATTTTATGGAGC |
| | | | TATGCCTACTGGTGTTACTGTTTCAGAAACTGGT |
| | | | CGTATTTTCATTTGTTTTCCGAAATGGGGAGACG |
| | | | ATGTTAAATTTACGGTGGCGGAGATAATTGGGGA |
| | | | TAAATTGCAGCCTTATCCTGATTTACAAACCAAT |
| | | | TTGGTTAACCCTGAGAATATCAAAATGACTTTCA |
| | | | TCAGTGTCCAAAGTGTAGTTACTGATGGAAGAGG |
| | | | AACACTTTGGGTACTAGATACGGCGGCACCCAAT |
| | | | TTTTCTAAACCAATTAAAGGCGGGGCAAAATTAG |
| | | | TCGCTGTTGATTTAAAAACCAATACAATCAGAAG |
| | | | AGTTTATACCTTTACAGAAGATGTTGTTCTTCCA |
| | | | ACAACTTACCTGAATGATGTCCGATTTGATTTTC |
| | | | GTGTTGGAACTGCAGGTTATGCCTATATAACGGA |
| | | | TTCTTCTTCCAAAGGACCAGGAGCTATTATCGTC |
| | | | GTGGATTTAGAAACCGGAAACGCATTTAGACGGT |
| | | | TAAATGGAGAGCAAATCCAACTTCACCGGATTCC |
| | | | TATTTTATACCGAAAATAG |
| Seq. | L-dopachrome tautomerase- | B. velezensis | ATGAATCGTAACAGGGATGGTTCAACTTCCCCGT |
| ID. 50 | related protein - gene | | TTAGGCTGGCATCCGACGGTATTGCGATTTCTCC |
| | | | TGACGGAAAGGTATTATTTTTTTGTCCACTGACC |
| | | | AGCCGTCATCTGTTCTCGATCTCAACAGAAGCCC |
| | | | TGAGAGATAGAAATATACAGGACATGGATTTACT |
| | | | TTATCATGTGGCGTATTGGGGAGAAAAAGGCGCG |
| | | | TCTGATGGAATGATTACAGATGCAAAAGGAGCCA |
| | | | TTTATGCCGGAGATTATGAGAACAATAGTATTCG |
| | | | AAAGATGCTGCCGAATGGCACAACGGAAACCATC |
| | | | GCACATGATCCGAGAATTTTATGGCCGGATACTT |
| | | | TTTCGATTGGTCCGGATGGATACTTATACGTCAT |
| | | | CGTGAACCAATTACATCGGCAGGCTAGATTTCAT |
| | | | TATGGAAAGGACTTACGACAGAAACCTTATAGTT |
| | | | TACTTCGTATCAAAATTGATGAATTTCCTGCTCC |
| | | | TGCTTTTTGA |
| Seq. | Terminase small subunit - | B. velezensis | ATGAAATTGACAGAGAAACAGAAGCGTTTTGCCG |
| ID. 51 | gene | | ACTTTTATATTGAGCTTGGTAATGCGACTGAAGC |
| | | | GGCTAGAAAAGCGGGTTACAGCCAAAAGACAGCT |
| | | | AAATCAATTGGTCAAGAGAACCTGACGAAACCTG |
| | | | ACATCAAATCCTATATTAAAGAGCGGCTTGCTGA |
| | | | AAAGGATGCGGAAAGGGTGGCTTCACAAGACGAG |
| | | | ATTCTTGAATTCTTAACGGCAGTGATGCGGGGCG |
| | | | AGAAGACTGAACAGATTCCCGTCGGCATCGGAGA |
| | | | AGGCGCTCAGGAATTAGAGGATAAAGACCCCTAT |
| | | | CTAAAGGATCGGGTGAAAGCAGCTGAACTGCTCG |
| | | | GTAAGCGGCACGGTATGTGGACTGAGAAAGTTGA |
| | | | TATGACAGGCGAATTCGCTGTGCAGTTCATTGAT |
| | | | GACTCAGGTGATGAAGAATGA |
| Seq. | Cell wall-binding protein | B. velezensis | ATGAAAAAACTATTCAAATCAATCATTTTATCAG |
| ID. 52 | YqgA precursor - gene | | CTGCATTACTTACGGGAGCGGTGGCCGTTGCGCC |
| | | | GTCTGCTTCCGCAGCATGGTCAGGCTGGCAAAAT |
| | | | GAATCTGGGTACAGCGGCCGGGTGTTTACGGATG |
| | | | CCACGACATACACGGCTGGCGCTTCCACGATAGA |
| | | | CTGGAAAGCCGAGAAAAAAGGTGATTCCGGCAAG |
| | | | AGGAACTATATCGGAACATTTGAATCAGCTAAAT |
| | | | TTCAAAGGGGGATTCGATGGCGCTGA |
| Seq. | YycA Aldo-keto reductase | B. velezensis | ATGGCTTTCCGCCTCTTTTTTCACGAAGAAAATT |
| ID. 53 | (AKR) superfamily - gene | | TTGGCAAAACAGTCTCACAGCTGAAAAAACTTGT |
| | | | TGACGAAGGAAAAGTGAAATATTTCTTATTATCC |
| | | | GGAAACAATTCAGGCAACACAAAGCTTGTCTCAT |
| | | | GGATTAAAAAGAACGGCACAAAAAACTCAGCTGA |
| | | | CCCATACAGCACCGAAAAAAACAAGCTCAGCCCG |
| | | | GCAAGGAATGACAGGCGGCCCGGGCGGACAAAGT |
| | | | CAGCAGACTCTGTATGA |
| Seq. | MarR family transcriptional | B. velezensis | ATGGTTAACTCATTGAAACTAGTTCAAAGAGCGG |
| ID. 54 | regulator - gene | | AATTATGGGATGAGCAAAATGAAAATATAGTTGA |
| | | | GAAGTTATACGTAGATCACTTACCAAACGATGGC |
| | | | CTACTTAGGGAAATTATACAGGATGATACTGTTT |
| | | | TTTTGGTGGGAAAAAAAACAGGAGAGTCCACGAT |
| | | | TTTTACAAGGGCCCAAGAATATTCTCGTATAAGT |
| | | | AAAAGTGAGATTTCCGCATATGTTGATGTAAAAA |
| | | | CAATGTACGGTAAGTCCCAAGTCAAAGAACTAAA |
| | | | TGAGGATTCAGATTATTGTAAAACCTTACTTTGT |
| | | | AATTGTTTTATTAAATCAAGTAATAAGCGACTTA |
| | | | TTAAAAGAAGTGAATAA |
| Seq. | DUF4181 domain-containing | B. velezensis | TTGAGTCTTTTACCATTAAACTCAGCCTTATGTC |
| ID. 55 | protein - gene | | TTATAGAATGGCGAAACTTACAAGCTCCTCACCC |
| | | | GTATGAAACGAGTGACACATTTTTAGGAGGCAGA |
| | | | CAGTTACTGATTTTTTCAGCCGTATTTATTTTGC |
| | | | TCAGCCTTATGTTTCAAGTATTTGCCGTCAAAGT |
| | | | TCTCAATTTGCCAAAGCCTGTTAAAAAGCAGAAG |
| | | | CCGATAAACGGCACACATCGATTAATCGAAATCA |
| | | | CACTGCTTATTACGGGCATCGCAGCGGTGAATTT |
| | | | TTTCAATGAAATGTACGTATTTGCGGGAGTTGTT |
| | | | TTCATGCTATTTTGCTGCACCGGCATAATGGAAT |
| | | | GGCGGCACCGGCGGGAGGAAAAGCAATATGTGTA |
| | | | CGATTCGTTTTACGCGGGTCTTTCTGCCGCGCTT |
| | | | GGCGTGCTTATTTTTTTCATCAGTTAG |
| Seq. | Phage integrase SAM-like | B. velezensis | ATGCATTCAGAAAATCGAAAAGGTAAGCGTGTGA |
| ID. 56 | domain-containing protein - | | AGGCCGGAAGGGCCTCGGTAAAGACAACGCGAAA |
| | gene | | AGGTACGTACGATCTCGAATACATATTCGATAGA |
| | | | CTGTGCGATGCCAAACGTGCTGAGAATGTCTTCA |
| | | | GGAAACGATTGATAAGTACATGCGTGCTTGTTGA |
| | | | TATTTGTCGGATTATGCTGAAGAGATCGGAGTGT |
| | | | CCGCAGACATACGTCATATTGACGTTGACTTTGC |
| | | | GCGCGGCTTTGTATCGTGGCTGA |
| Seq. | isocitrate/isopropylmalate | B. velezensis | ATGAAAACATACAAGATTGCCAGCATACCGGGGG |
| ID. 57 | family dehydrogenase - gene | | ACGGTGTAGGGAAAGAAACAGTTCCGCAAGCGGA |
| | | | ACGGGTGTTGAACGCAATCGCCGAATTACATGGA |
| | | | GGCATTGCCTTTGATTTCAAATGA |
| Seq. | hypothetical protein - gene | B. velezensis | TTGCTGAAAAGGTTTTTTCTTGTTTTTCTTACGC |
| ID. 58 | | | TGATAATGTCTATATTTGCTGTATTCGCGGCTGC |
| | | | TGAAGAGTCTCCCGGCGATCGTGTCATTTCTTTT |
| | | | GACGAGCCTATGATTGTATTGATCTCACTCGGAA |
| | | | TCATCTTATGTTTGTTCATTCCGCCATTCGTCAT |
| | | | GTCCTTTTTTGGCAACTTGGTTGTGAGAATCATA |
| | | | TCCGGGAGTTTATCAAAGTTTTATCGTCCTCACG |
| | | | TTTGGGGGGCTCATTCCGATTGGATTTCTGATTC |
| | | | CCAATGGCTTTTTGACAATTCTTGCTGCCGTCCT |
| | | | GGGAACTTTGGTCAGTTCAGCCAGTGTTGCGGCC |
| | | | ACCTTATTCGTTGGCAGGACAGAGAACACAAGTA |
| | | | A |
| Seq. | DUF4573 containing domain - | B. velezensis | GTGGCTCCTGGTTCTCCCGTTGCTCCAGTTGGTC |
| ID. 59 | gene) | | CTGTTACACCAGTTATTCCCGTTACACCAGTTGG |
| | | | ACCAGTTGCTCCGGTTGAACCTGTTACTCCTGTT |
| | | | GCGCCGGTTTCTCCTGTTGCTCCTGTTGGACCCG |
| | | | TGATTCCGGTTGGGCCAGTTTCTCCAGTCGCCCC |
| | | | AGTTACTCCGGTTGGTCCCGTTTCTCCGGTCGCC |
| | | | CCTGTTACTCCATTTGCTCCAGTTATACCCGTTG |
| | | | CTCCAGTCACTCCGGTTGCTCCCATGGCTCCGGT |
| | | | TGCCCCGGTTACTCCTGTTGAACCCGTGATTCCT |
| | | | GTTGGCCCTGTTGCTCCAGTCGGCCCTGTTGCTC |
| | | | CAGTCGAGCCGGTTGTTCCCGTTACTCCTGCTAG |
| | | | GCCGGTTTCTCCAGTAGCTCCGGTTTCTCCGGTT |
| | | | GCGCCAGTAACTCCAGTTGGACCGGTTGGGCCTG |
| | | | TTGGCCCGGTTGGACCTGTTATTCCGGTTGAGCC |
| | | | GGTTACACCAGTCGCCCCAGTTGGGCCGGTTGAT |
| | | | CCTGTAATTCCGGTAGGGCCTGTTACTCCAGTTG |
| | | | AACCGGTTTCTCCTGTTACACCCGTTGGACCTGT |
| | | | TCCCCCGGTTGCGCCTGTTTCTCCAGTTGGGCCG |
| | | | GTTGGACCGGTTGCACCAGTCGAGCCGGTTGGGC |
| | | | CTGTTACTCCAGTCATTCCGGTTGTTCCCGTGGT |
| | | | TCCGGTTGCTCCCATGGCTCCGGTTGCCCCGGTT |
| | | | TCTCCTGTTGGACCCGTTTCTCCCGTTGAACCTG |
| | | | TTGCGCCGGTTAGGCCAGTTTCTCCAGTTGCACC |
| | | | GGTTACACCAGTCGAACCGGTTGAGCCTGTTACT |
| | | | CCAGTCGCTCCTGTTATCCCCATTGACCCAGTCA |
| | | | CTCCCGTTACACCGGTTGTGCCGGTTGGACCGGT |
| | | | TGCTCCTGTTGAGCCAGTTTCTCCGGTTGAACCT |
| | | | GTCACTCCCGTTGAACCAGTTGACCCTATTTCTC |
| | | | CGGTTGGACCCGTTGCCCCAGTTGGGCCGGTTGC |
| | | | TCCGATTGAACCTGTTGCTCCGGTTGGACCGGTC |
| | | | TCTCCCGTTGGGCCGATTTCTCCAGTTACACCGG |
| | | | TTGCGCCGGTTTCTCCCGTTGAACCAGTTGAGCC |
| | | | GGTTGCTCCGGTTATTCCTGTTGACCCTGTTACA |
| | | | CCGGTCCCTCCGGTTTCTCCCGTTACACCCGTTG |
| | | | AACCGGTTGGGCCGGTTGGGCCGGTTGAGCCAGT |
| | | | CGCCCCAGTTACTCCTGTTGCTCCAGTTATACCC |
| | | | GTTGCTCCAGTCACACCCGTTGGACCAGTCACAC |
| | | | CCGTTGGACCAGTTGCGCCTGTTGATCCGGTTGA |
| | | | GCCCGTTATTCCGGTCGCTCCCGTTGGTCCGATT |
| | | | TCTCCAGTTACACCGGTTGCGCCGGTTTCTCCCA |
| | | | TTGAGCCAGTTGAGCCGGTTGGGCCGTTTTCTCC |
| | | | CGTTGCACCAGTTACTCCGGTTGCTCCAGTTTCT |
| | | | CCTGTTGGGCCAGTTTCTCCAGTTACACCGGTTA |
| | | | CACCAGTCGAGCCGGTTGTTCCTGTTACTCCAGT |
| | | | CATTCCGGTTGGACCGGCTTCTCCAGTAGCTCCG |
| | | | GTATCCCCGGTCGAGCCAGTTTCTCCGGTTGCTC |
| | | | CAGTTACTCCCGTTGCCCCTGTTACTCCGGTTGA |
| | | | GCCAGTCGCGCCAGTTTCTCCAGTTGAACCTGTC |
| | | | ACTCCTGTTGATCCGGTTGGACCCGTTGCCCCTG |
| | | | TTATTCCGGTTGAGCCTGTTGATCCGGTTGGACC |
| | | | GTTCTCTCCCGTTGGGCCGATTTCTCCAGTTACA |
| | | | CCCGTTGCCCCTGTTGGGCCGGTTTCTCCCGTTG |
| | | | GGCCGATTTCTCCAGTTACACCCGTTGCCCCTGT |
| | | | TGGGCCGGTTTCTCCCGTTACCCCAGTTACTCCG |
| | | | GTCGCTCCAGTTACTCCCGTCGCCCCGGTTACAC |
| | | | CAGTCGAGCCGGTTGTTCCTGTCGAGCCAGTTTC |
| | | | TCCGGTTGCTCCAGTCGGTCCTGTTGCTCCAGTT |
| | | | ACTCCCGTTGCACCGGTTACTCCTGTTGAGCCCG |
| | | | TGGTTCCGGTTTCTCCGGTTGGCCCTGTTACACC |
| | | | GGTTTCTCCGGTTGAACCCGTTGTTCCGATTGGG |
| | | | CCGGTTTCTCCTGTTATCCCCGTTGCTCCAATTG |
| | | | GGCCGGTTTCTCCTGTTATCCCCGTTGCTCCAGT |
| | | | TGGGCCGGTTTCTCCTGTTATCCCCGTTGCTCCA |
| | | | GTCGGGCCGGTTTCTCCTGTTATCCCCGTTGCTC |
| | | | CAGTTGGGCCGGTTGTTCCCGTTCCCCCGGTTAC |
| | | | ACCAGTCGCTCCGGTTGGGCCGGTTGACCCTGTT |
| | | | GCTCCTGTTACTCCAGTTGAGCCCGTTGCGCCAG |
| | | | TCGATCCGGTTACTCCCGTCGCTCCAGTCGATCC |
| | | | GGTTACTCCCGTCGCTCCAGTTTCTCCTGTTGAA |
| | | | CCAGTTACACCAGTTGCACCGGTTTCTCCGGTTG |
| | | | AGCCAGTAATCCCTGTTGCTCCTGTTGCTCCTGT |
| | | | TGTTCCGGTTGAACCAGTTGAGCCGGTTTCTCCG |
| | | | GTTGCACCTGTTGCTCCAGTCACTCCAGTTGCAC |
| | | | CGGTTTCACCTGTTGAGCCAGTAATCCCTGTTGC |
| | | | ACCGCTTTCTCCTGTTGAACCAGTTACACCAGTT |
| | | | GAGCCCGTTGCTCCTGTTGTTCCCGTTACACCAG |
| | | | TTGGACCAGTTTCTCCGGTTGGGCCAGTTTCTCC |
| | | | AGTTATTCCCGTTGCTCCGGATGCACCCGTCGCT |
| | | | CCAGTTTCTCCTGTCGATCCGGTTTCTCCGGTTG |
| | | | ACCCTGTTGCTCCTGTTACTCCAGTTGAGCCAGT |
| | | | CGCTCCAGTTACTCCCGTTGGACCAGTTACACCC |
| | | | GTCGATCCTGTATCCCCGGTTGGCCCGGTTTCTC |
| | | | CGGTTATACCAGTTGGCCCTGCTGCTCCTGTTGG |
| | | | GCCAGTTACTCCCGTTGAACCAGTTGATCCCGTT |
| | | | ACACCAGTTGGACCGGTTGATCCGGTTGATCCGG |
| | | | TTGCGCCTGTTGGGCCGATTGCACCCGTTGCACC |
| | | | GGTTGATCCTGTTGATCCTATTGCCCCAGTTACT |
| | | | CCTGTTGGGCCAGTTTCTCCCGTTGCCCCAGTTA |
| | | | CTCCAGTTGCACCGGTTGATCCTGTTACCCCAGT |
| | | | TGGGCCTGTTGTTCCTGTTGCTCCAGTTATTCCA |
| | | | GTGGCGCCCGTTGCGCCGGATGCACCTGTCGATC |
| | | | CGGTTTCTCCTGTTGATCCAGTTACTCCGGTTGG |
| | | | GCCTGTTGAACCTGTCGATCCCGTTGCTCCGGTT |
| | | | GGGCCTGCTGCGCCAGTTGTGCCAGTTGCGCCGG |
| | | | TTGAGCCGGTCGCACCAATTGAGCCGGTCAATCC |
| | | | AGTTGGACCAGTCACTCCGGCTGGGCCAGTTGCG |
| | | | CCGGTTGAGCCAGTTGGCCCGATTTCTCCAGTTG |
| | | | AACCGGTCGCTCCTGTTACTCCGGTGAACCCCGT |
| | | | TGGTCCAGTCAGCCCCGTTGCTCCGGTTGAACCT |
| | | | GTTACCCCGGTTGTCCCCGTTACACCAGTCGCTC |
| | | | CTGTATCCCCGGTCGGACCTGTTATTCCGGTTGA |
| | | | CCCGGTTGCCCCCGATGCACCTGTTACTCCGGTT |
| | | | GGACCTGCCGCTCCGGTTGAGCCTGTTTCTCCCG |
| | | | TCGCCCCGGTTACCCCTGTTACTCCTGTCGATCC |
| | | | CATGACCCCCGTCGGGCCAGTCACACCAGTTGAT |
| | | | CCGGTTAACCCCGTTTCTCCGGTTGAACCTGTTA |
| | | | CCCCAGTTGTCCCAGTTACCCCAGTCGCTCCTGT |
| | | | ATCCCCGGTTGGACCGGTTGCTCCTGTTGGACCT |
| | | | GTTACTCCGGTCGCTCCGGTGAGCCCGGTTACTC |
| | | | CCGTTACTCCTGTTGCTCCCGTTATCCCAGTTGC |
| | | | TCCGGTTTCTCCGGTCGAGCCGGTTGCACCCGTT |
| | | | GGACCAGTTACACCAGTCGCCCCAATTGGGCCAG |
| | | | TCACACCAGTTGGGCCTGTTGCTCCAGTTGAGCC |
| | | | CGTTGAGCCAGTCGCTCCTGTTTCTCCTGTCGAT |
| | | | CCCGTTGGACCAGTCTCCCCAGTTGAACCGGTTG |
| | | | ACCCCGTTTCTCCTGTTGATCCGGTTACACCAGT |
| | | | TGACCCCGTGACCCCAGTCGGGCCAGTCACTCCT |
| | | | GTTTCTCCTGTTGATCCTGTTGATCCTGTTGCCC |
| | | | CTGTTACTCCTGTTGGGCCAGTTTCTCCAGTTGG |
| | | | CCCAGTTACACCCGTCGCTCCTGTCTCCCCGGTC |
| | | | GGACCTGTTATTCCGGTCGCTCCTGTTGGGCCGA |
| | | | TGGGGCCAGTAGCCCCTGTGGCTCCCGTTATCCC |
| | | | CGTTGCCCCAGTTACTCCAGTTGACCCCGTTTCT |
| | | | CCTGTTGATCCCGTTGCTCCTGTTACTCCGGTTG |
| | | | AACCTGTTACCCCAGTCACTCCAGTTGAACCGGT |
| | | | TGCTCCCGTCGATCCCGTTGCACCCGTTGAGCCG |
| | | | GTGGCTCCTGTTACTCCAGTTGAGCCCGTTGAGC |
| | | | CAGTCGCTCCAGTTACTCCCGTTGGGCCAGTTAC |
| | | | ACCAGTCGCTCCTGTATCCCCGGTTGGACCAGTT |
| | | | GCTCCTGTTGCTCCAGTTACTCCCGTTGAACCAG |
| | | | TTGATCCCGTTAAACCAGTTGATCCCGTTAAACC |
| | | | AGTTGATCCCGTTAAACCAGTTGGACCGGTTGCT |
| | | | CCGGTTTCTCCTGTTGGTCCTGTTGCACCAGTCG |
| | | | AGCCGGTTGTTCCCGTTCCCCCGGTTACACCAGT |
| | | | TACCCCAGTCGATCCTGTCGATCCCGTTGCACCC |
| | | | GTTTCTCCTGTTGGACCGTTTTCTCCCGTTGGGC |
| | | | CGGTTGAGCCAGTTACTCCTGTTGCGCCTGTTGG |
| | | | TCCTGTTGGTCCGGTTATCCCTGTTGCACCAGTT |
| | | | ACACCGGTCGCTCCGATTTCTCCTGTTGCTCCAG |
| | | | TTACTCCGGTCGCACCAGTTGATCCCGTTGCACC |
| | | | CGTTGAGCCGGTTGCTCCGGTTGGGCCGATCGGG |
| | | | CCAGTAACCCCTGTGGCTCCCGTTATCCCCGTTG |
| | | | CCCCAGTCACTCCAGTTGATCCGGTTACACCAGT |
| | | | TGGACCCGTTTCTCCCGTTGAGCCCGTTGCTCCG |
| | | | GTTGTTCCTGTAATTCCAGTTGGGCCCGTTACAC |
| | | | CAGTTGGACCGATCTCTCCCATTGGCCCGGTTTC |
| | | | TCCTGTTGAACCAGTTGAACCAGTTGACCCTGTT |
| | | | GCTCCGGTTGGGCCTGTTCTACCAGCTGAGCCTG |
| | | | TTACTCCCGTCGACCCGGTTACTCCAGTTGGACC |
| | | | GGTCGAGCCGGTTGGGCCGGTTGCTCCAGTTGAA |
| | | | CCGGTCGATCCCGTGACCCCCGTCGGGCCAGTCA |
| | | | CACCAGTTGCTCCGGTTATGCCCGTGGCGCCCGT |
| | | | TGCGCCGGTTGATCCGGTCGCACCAATTGAGCCT |
| | | | GTCAATCCAGTTGGACCAGTCACTCCGTCTTGGC |
| | | | CAGTAACCCCGGTTGCCCCTGTTACTCCCGTCGA |
| | | | CCCGGTTACTCCAGTTGGACCAGTTACTCCAGTC |
| | | | GGGCCGGATGCACCCGTTGATCCTGTGACTCCTG |
| | | | TCGCTCCTGTTGTACCAGTTGAGCCGGTTGTACC |
| | | | TGTGGCTCCGGTTGAACCAGTTCCTCCGGTCACT |
| | | | CCTCTTGGTCCGGTTACTCCTGTGGGGCCCGTCA |
| | | | CTCCTGTGGGACCTGTAATTCCCGTTACGCCAGG |
| | | | TTCACCCCCGGTTTGCGCCAATGAAACATCGTCT |
| | | | ATTAAAATCGTGCCGGTAGAAACTTTACTTAATA |
| | | | CAATGACGGCAGAGGCCGCGCCAGAAGGCACACT |
| | | | GCTTGTTTTATAG |
| Seq. | restriction endonuclease | B. velezensis | ATGTTAAATGATTATATAAAGCCCAGTATTTTAT |
| ID. 60 | subunit S - gene | | GGACAGATGTAGAGGATAGAATAGATGCAACCTT |
| | | | TTATCGGACGGACTTTGTAGTGAGTCTTGATAAG |
| | | | ATATTTAAGAATTTAACTATTCCTGTGTTGCCTA |
| | | | ATAGGCAGTTGATACATGAATTAACAGATTATAC |
| | | | TGCAAATGGAAGTTTTGAATCATTGAAAAAAAAT |
| | | | GTCAGATATACAAATGAAGCAGGAATACCTATGA |
| | | | TTAGGGGTAAAAATTTGAAGAATGGTGTTTTAGA |
| | | | TAAAACTGATCTCAATTACGTCACAGATGATACA |
| | | | TATAGATTCTTGAAAAAAAGTGAATTAAACGCAG |
| | | | GTGACCTATTATTTACAAAAACTGGAACCCTCGG |
| | | | GAATACTATTGTTTGGTCTGAGTATTTCGGAAAG |
| | | | GCAACGTTAGGTGACAATATATTTAAAGTTAGAT |
| | | | ATAAAAATGGAATCAACCCTTATTATATTCATGC |
| | | | GTATTTTTCTACTGATTATGGCTATAAATGGGTA |
| | | | GAAAGATTTGCACAAGGTGGGGTGCAACCAACTA |
| | | | TTATTTTAGCATTAGTTAAGTCAGAAATTTCGGC |
| | | | TGAGCCTTTGGTAATTCCTCTGCCTAACACTTCT |
| | | | GATCCTTTATAG |
| Seq. | N-6 DNA methylase - gene | B. velezensis | ATGCAAGCATTAATTGAAGAGTTAAAAGAACAAC |
| ID. 61 | | | TCATGATGAAGGGGTATAGTACATTTTCTGGACC |
| | | | AGAAACACATGAAGAAGATGGCACAACGATTTTT |
| | | | TTAGCTAGTGGTGATGAAAAAATTGTTTATGTAA |
| | | | AGGTATTTGACAAAGGAGCAGATTTATCTTTTGA |
| | | | AGTGCAAGATGCTGCTTTAATAGAGGCGCAGTAT |
| | | | CATGAAGAGGCTCCGGATTTCATTTGGGCATCAA |
| | | | ATGGTGAAGTGAATTATTACGCTGACTGTATTGA |
| | | | GTCAGATCCACTGGCAGAAATACCGGCCGTTATT |
| | | | GATGTAACAAAAAAAGAAAAACGACAAATTTCTA |
| | | | AACAAGAAGAATGGTCAAGAAAGATGTACTCTAC |
| | | | TCTTCAAAAGCGATTTGATGATTTGCATGAGCGC |
| | | | TTATATGGACCGGGTGGTGCTGATAATATCAGCA |
| | | | GTACGAACGAAGCCATTGATGAATTAAGTAAGTT |
| | | | GCTTTTTATGGAGACTTTCCGCTTGTATCATCCA |
| | | | GACTATGTATTAAGAGAAGGAGAAAATGCAGGTC |
| | | | TGCGAATTTTGGATATTTTAAAGTATCAATACAT |
| | | | AAAAGAGAATGGAAAAGAGGCTGTTAGGCAAATT |
| | | | CGTGATGCTTTTAAAGAAATAAAAAACCATGAGG |
| | | | ATTATGTTGCGGTAAATGATCTTGGTGAAGAATG |
| | | | TTTTATTTTCAGTGAAGATGAATACATTCGTTTA |
| | | | GAAAATCCCGCTAACTTTATTGCTGCTTTTGAAA |
| | | | CGTTTCAGGAATTAGATGATTTTGAAGATGAAAA |
| | | | TGGAAACCGAGTGAAAGCCACATTGAAAAATGTC |
| | | | AGTGCGGATATTCTAGGGCGTTTATTCGATGTAT |
| | | | TGATGCGTGGTAAATATGATAATAAAATTGGTCA |
| | | | AGCTACTTACTTAACACCGAGACAAGTAACTGAA |
| | | | GCAATGATTGAAATGGTTGTGCATGATTTAACAT |
| | | | CTAACCCAAAAGAAGCATCAAAATTAATCACAGC |
| | | | TGATGAAAAAGGTATTCCTACATTTAGAATGGGT |
| | | | GACATAACTTGCGGCTCCGGTGGATTCTTAATAA |
| | | | AAGGTTATGAAGCAGTAAAAAAATATATGCTCAC |
| | | | CCAGTTTACGGGAACGATGAAAGAAAAAATGGAG |
| | | | AAGCATTTTGAACTTATGAAAGAACATAGCTTTG |
| | | | TTGGAGCAGATAAGACACGAAACATGGTATTAAA |
| | | | AGCACGCTTAAATATGGCGATGCATGGATTACCA |
| | | | AAAGCGCCAATTTTCTGGGTAAATGATTCATTAA |
| | | | TGACTGAATCATTAAAACCGAAGAGTTTTGATTT |
| | | | AATAGTTACAAACCCACCTTTTAAATCAGGATCA |
| | | | GTTTCAAGTAAAACTGATGAGGGGAAAGCTATAT |
| | | | TAGAACGCTATAGCGAGGGAATTGACGAAACAGG |
| | | | CAAACCTAAAAAAGAGGGTCTTTGCTTAGGTGCA |
| | | | AAACCTAATCCAAAAGGGATTTGGAAAGAAGTAA |
| | | | GTTCGACAGACCAAGCAGTTTTGTTTATTGACCG |
| | | | TAACTTACAGTTGTTAAAGGATGGGGGACAATTA |
| | | | ATAATCGTCTTACCAGATGGGATATTGAGTAACT |
| | | | CAGGTTACAAACACGTAAGGGAATATTTAATGGG |
| | | | TAAAAAAAATGAAGTAACAGGTGAGTTTGAAGGA |
| | | | GGTAAGGGGATAGTTAAGGCTGTAGTTTCACTCC |
| | | | CGACAGTAACATTCCAATTATCAGGAACCGGAGC |
| | | | AAAAACATCTTTTCTATATATTAAGAAAAAAGAA |
| | | | CATGAAAATGAAAAACAAGGTCCTGTATTCATGG |
| | | | CAGTGGCAGAGGAAATTGGGTTTGATGTGAAAAA |
| | | | TAAAATAGAAGTTCAGCTGGGGAATGATCGGAAT |
| | | | GATTTATTGAAGATTGTTGATGTTTATAAAAAGG |
| | | | GAGTTAATTAG |
| Seq. | hypothetical protein - | B. velezensis | ATGAGATTGATATTACATGAATACCTAACCATAC |
| ID. 62 | gene | | CAAAAACAACAGAACAATTAGTGAACATTGCAAA |
| | | | AGAACATGGATTGAATTGGAATGAGGAACAAGTA |
| | | | AAATTATATTGCTTGCTTGATCCCTCTATATATG |
| | | | AAACAGAATCAGGTCAATGGGCTGTTAAGGTTGA |
| | | | TGACCGTCGTCATACCATATTAGGAGCAATTGAA |
| | | | AAAGCATTAGAAAAACGGCCGATGACTAAGATTG |
| | | | ACCCCAATATTATTGAACAGTTACCGAGTGATAT |
| | | | GATTGTGCCTCATAATGAGGTTATTGAAGTAGCT |
| | | | GTTAGTACAGGGCGTTATGAATCACCGAGAGAGA |
| | | | ATATTATTCGCGTGAAACGAAAATAA |
| Seq. | class III lanthipeptide - | B. velezensis | ATGAAAGAGGTGCTAGTACTTCAACAATTTGATG |
| ID. 63 | gene | | AAAAGTCTGAAGTAGAGCCTACGGCATGGACTGT |
| | | | TACTCTAACAACTATTTCAGTTACATTATCTACC |
| | | | CTTAGCAATCATTGCTAA |
| Seq. | class III lanthionine | B. velezensis | ATGGACAAAAAATTAGTTTATTTCCAACACATGA |
| ID. 64 | synthetase LanKC - gene | | GTGAAGATTCAAAATACTATATAGAGAAAAGCCC |
| | | | AAATGCGGAGACTGATCCATACGAAGTACATAAT |
| | | | ATCCCGAATCACTATACTGTTAGTACAGAATATA |
| | | | AATCACCGTGGAAGTTTTATAATAATCCAAATCA |
| | | | TAACCTTAAAGAACAAGGCTGGAAAATACACGTA |
| | | | AGTGCGACAATGAATAATGCTCAGGATATATTGA |
| | | | AAGTTGTAAGTCAAATTATGTTCAAACATCAGAT |
| | | | CCCATTTAAACATATTAAAGATAAACAATCTCTA |
| | | | TTTAATATGAATTCAAAGCATGGTAATCGAACTT |
| | | | CATCTGGAAAATTTATCGCTATATATCCTTCCTC |
| | | | TAATAACATGTTCTTCGATTTATTAAATGAACTT |
| | | | TATGAAGAATTAAAACATTATGAGAATGGGCCAT |
| | | | ACATACTGAACGACAAATCGTGGAAGGATAGTAA |
| | | | TATATATTACCGATACGGTGCATTTAAGAATATT |
| | | | TACAATGAAAGAGGAGAACTTTGCATTAAAGACG |
| | | | AAAACGGAAATTTAATTCCAGATAATAGGAATCC |
| | | | ATATTATAAAATACCTGATTTCGTAAGTGATTTT |
| | | | GATCAGTTTTTAGATACCAAGAATAACTTATTAG |
| | | | ATACTCAAGATGTAAGTAATTTTAATGATTATGA |
| | | | GTTCCAAGGAGTCCTTAGGTTTAATAATGGTGGC |
| | | | GGTATATATACTGGAATAAGGAAATCAGATGGAA |
| | | | AAAAAGTAGTAATCAAAGAAGGAAGACCTAAAGT |
| | | | AGGGTTAGATGGTCAATATAATGATGCAATTGAT |
| | | | AGATTGAATATAGAATACGAAGCATTAAATAAAC |
| | | | TGACAGAAGTTGAAGGTGTTGTTAAAGCGCTTGA |
| | | | TTATTTTAAAGCGTGGAAGCATGTGTTTTTAGTT |
| | | | GAAGAGTTTGTAGAAGGTGTAACTCTTCAATCAT |
| | | | GGTGTGCAATCAATTATCCGTTCAACGTTGAGAC |
| | | | TGATAAAAACGAATACATTAGTAAGGTAAAAAAA |
| | | | ATAATTAACAGCTTGATAAAAGCAATTAAACTAA |
| | | | TGCATAGTAAAAATGTTGGTATGGGAGATTTGCA |
| | | | ACCCCAAAATATAATGATTACCCCTGAATTAAAT |
| | | | GTTAAGATTATTGATTTTGAGGCTGCTAGTGCTC |
| | | | TCTCTGAAGAAAATGGAGCGTTGCAAACAATTGG |
| | | | CTACACACATAAGAAAAATAAAAATCATAAGGAG |
| | | | CGAGATTGGTATGCACTAAAAAAAGTTTTAAGAT |
| | | | TTTGTATTCTGCCAATAGGTCCAGTTGTTGATAT |
| | | | AGAAGAAACTATAAAAAAGAAACATGATATGTGG |
| | | | ATAGAGGAGGAGTTTGGCAAAGATGTCTATCAAT |
| | | | ATGTTGTAAACACAGAAAATCTATGCGACAAATA |
| | | | CTTATCTGATACCAAAGAAAAGAGATTCAACAAT |
| | | | AATTATGACAGTAAAATACAAAAAGAAATGGACA |
| | | | TGTCAAAAATTATTAAAGGATTGAGAAAAAATAT |
| | | | AGAGAGTAATATTTTACCCAATAGCTCGCTAATT |
| | | | CATGGCGATGTACGCCAATTTGTAATGCCATCTG |
| | | | GAAAGTTAAATGTACTAACTGGAGGAACTGGAGC |
| | | | TGCATTGGCATTGTACAGATCTGGAAACATCAAC |
| | | | AAGCGAGTTCAGGATTGGATTGAGATTGTTTTAC |
| | | | TTGAAAATTTGAATGTACTTAATGATGATATTGG |
| | | | GTTATTTACAGGAAAAGCTGGTATCGCAGTCACA |
| | | | TTGTATGAATTGGGATACACAGAGCAAGCTGTGG |
| | | | ACATATTCAATAATCTGTCATATGAAGATATGAA |
| | | | AGATATTTCATTAAGGTCAGGACTGTCGGGAATA |
| | | | GGTCTATCACTAATTAGCTTATACTTGGAAGAAA |
| | | | ATAATGATGATCTTTTGGTTACTTCTGAAAAAAT |
| | | | TGCTGATCAGATAGAAAATAGGATTATTGAAGAT |
| | | | GATGAATTATCAGTTACTGATTTTGCAGCAGTAC |
| | | | CTATAGGTTTGATTGATGGTTGGTCAGGAATATC |
| | | | TTTATTCTATGCGGCATTATATTCAGTCACTAAC |
| | | | AATGAAAAATATTATTTGAAATCAAAAGAATATA |
| | | | TTGAGCGAGATTTAAAACACTGTAAGGTAGATGA |
| | | | AGAGACGAATACTTTGCAAACTTATGATGATCAA |
| | | | AACCCTCGATTATTACCCTATTTATCTGGGGGAT |
| | | | CAATCGGAATAGGGATTGCTATTTGGTATTTAAA |
| | | | TTATGTAAGAGACACTAATTTATTTAGAGAAGAA |
| | | | TTAGAGCAAATAATCAATTTAAATAAAGTAAGGA |
| | | | TCGCATATAGCGGTGGATTGTTTGATGGGATCGG |
| | | | TAGTTTCTTGTTAATCCCGCCAATGACTAAGGAA |
| | | | AAAAACGAAATCAGTCAGCATATTGAGCTGGCAA |
| | | | TGAAAAAACTAGAATTGTTTCTTATCCGTAAAGA |
| | | | TGATCAAATATTATTCCCTGGGAATTTCAGTTTT |
| | | | CGTTTAGCCGATGATTACTACTCCGGAAGTGCCG |
| | | | GCATAATATTGGGCATAAACTCTATCCAGTTTTC |
| | | | AAATCCGTTATATTGGTTGCCAATAATAAAAATT |
| | | | GATAGTTTCTTAAGAAAGACTAAAAAAGAATTGT |
| | | | TATTAAATAATTAA |
| Seq. | ABC transporter ATP-binding | B. velezensis | ATGATGACCAGCCCAAAATTTAAATCGATAGAGT |
| ID. 65 | protein (closest BLAST | | TTTTCTTCTTACTGAGAAAAACCAGACCGAATAT |
| | homolog; 100% alignment | | AAGACTATTTATATTGGCAATATTTTTAAGTGGA |
| | coverage; 96% sequence | | TTAAGCGCAATAGCTTCTACCCTAGTGCCTCATT |
| | identity; B. vallismortis) | | ATTTAAAAAGTCTAATAGACAATTATAATATTCT |
| | | | TGATAAGATCAATTTGCGGGATCTTTTAGGACTA |
| | | | TTAGGGATATTTTTGAGTAGCGGAATAATTGGAG |
| | | | CAGTTGGTAGTTATATGTTAAGTGTAGTAGGATA |
| | | | TAATGTTGTCGCAAATCTCAGAAATTTATTATGG |
| | | | GAAAAGATAGTTATACTACCTGTAGAGTTTTACG |
| | | | ATAAGAATCGTTCTGCGGACATAGCAAGTAGATT |
| | | | TGTTAATGATACTACGGTGATTTATAACTTAGTA |
| | | | AGTAACTCTTTTTCGCAATTTATCAATGCTATAT |
| | | | TAATAGTGCTTTTTTGTGGATTTTGGTTATTTTA |
| | | | CTATGATTGGCAATTGACACTAGTGATTATAGTA |
| | | | GCAATTCCAATTTATTTGTTGATTTTTATACCTT |
| | | | TAGGAAGAATTTTATCTAAATTATCTAAAATGAC |
| | | | ACAAAGCTTAACAGGAAAATTAAATGTAAGAGCT |
| | | | GTTGAAACAATATCCTCAATAAGACTAATTAAAT |
| | | | CTTCAACTGCAGAAAAACTACAAGTGGAAAAAGG |
| | | | AAGAGATGATATTGAAGCTTTAAGAAACATAGGC |
| | | | CTAAGGCAGACAAAATGGATGGTTATTTTAAACC |
| | | | CTATCATTAATTTAATGATGATGCTTATCATTAT |
| | | | CAGCATCATTGGATTCGGAGGGGTTAAATTAGCC |
| | | | AATGGAAGTTTATCGCCAGGAACGTTTATAGCAT |
| | | | TTCTAACCCTTATGTTCTATATCATAGGTCCACT |
| | | | TAATAATTTGGGGCTATTCTTTACTCAAATACAA |
| | | | AAGACGATAGGTGCAACAGAAAGAATTTCGCAAT |
| | | | TGATATATGAGCAAGGAGAAGATCAGCATAAAGG |
| | | | AAAAGAATTAGCGGAAACCTATAAAGATCTAGTG |
| | | | ATTGAAAATTTATCATTTTGCTATGGTAAAGAGG |
| | | | ATAAAGCATTTTCCTTGAACAATATTAATTTAGT |
| | | | AATAAAAAGAGGAAGTGTTAATGCCTTAGTAGGT |
| | | | CCTAGTGGTAGTGGTAAAACCACAATCATCTCTA |
| | | | TTCTGGAAAGATACTACAAGCCGAATAGCGGAAG |
| | | | TATTTATTTTGGTGAAGAGAATATTGAGTTATAT |
| | | | TCATTAAAATCATGGCGTAGCCAAATAGGATATG |
| | | | TAGCTCAGAATCATGATTTAATAAGCGGTACTGT |
| | | | TAGAGACAATCTCATCTTTGGTTTAGAAAATACC |
| | | | CCAAATGAAAACAGAATAGTGGAAGCATGTAAGA |
| | | | AGGCATATGCATGGCAATTTATAAGTGAATTACC |
| | | | TAACAGTTTAGAAACTAATATTGGAGAAAAAGGT |
| | | | CTGAATTTATCAGGGGGACAACGACAAAGAATTG |
| | | | CTATTGCGAGAATGTTTCTGAAGAATCCAAATAT |
| | | | AATCTTTTTGGATGAAGCCACTGCTAGTCTTGAC |
| | | | AGTCATTCTGAACAAATTGTGAACGAAGCAATGA |
| | | | AGAAGCTCATGAAGGATAGAACCGCTATTATAAT |
| | | | AGCACATAGGTTATCCACTATTATAAACGCTGAC |
| | | | AATATCATTTTTATGGAAAACGGTGAGGTAACAG |
| | | | GCCAAGGGAATCATTGGGAGTTAAAGCGCAGTCA |
| | | | TAAATTGTATCATCAATTCTGTGAGCAGCAGTTT |
| | | | AAAGAAGATAGTACCGATCCATTAGTATTAACCA |
| | | | ATTAA |
| Seq. | S8 family serine peptidase - | B. velezensis | ATGAAGAATTTAAGAAGAATTTCGATGGTTTTCA |
| ID. 66 | gene | | TACTTTTATTTGTATCCAGCTTGCAACCTATTAG |
| | | | CTCTCATGCTGAAGAATCAAATTATGATGAATCT |
| | | | ATCATAATTAACCTCGACTCAAAAGTAAGTGAAT |
| | | | CGACAATAAACAAGGTTAAGGATAGGTATTCCAA |
| | | | TATAGGAACAAACTATATTAAAGAATTAGATTTA |
| | | | TTGGTAATAGATAATATAAAACCAAGCCAATTTT |
| | | | CAGCAATCAAAGATATTGCTAAAGAAAAATTAGA |
| | | | CGCAAAAGAAATTATTGAGGATAAAGAGATTTCT |
| | | | CTTCCTAGTAAACAGCTATCTTCTAAATTGGCAA |
| | | | TTTCATCACAATTCACAGAAGATAACGACACTCT |
| | | | TTATGACAGTTGGTTATGGAATATCAAGCAAGTA |
| | | | ACAAATGATTATCGAAGTTATCGTATAAATAAAG |
| | | | GATCTCATAATACAAAAGTAGGAGTAATCGATTC |
| | | | AGGCATAGATTTCAATCATCCAGACCTCAAAAAT |
| | | | AATATTGTAGATACTGGAGAGTCTTTTGTTCCAG |
| | | | TTGAAGATAATACACAAGACTTTTATGGGCATGG |
| | | | AACTTCTGTGGCTGGGATAATTGCTGCTAACGGG |
| | | | GATGTAAAAGGAGTTGGACCAAATCTTGGTTTAG |
| | | | TGCCTTATAAAGTATTCGATAAAAATGGTAAATC |
| | | | TAAAACTAGCTGGGTTATAAGCGCAATTGTTAAA |
| | | | GCTACGGAAGATGATATGGACGTAATTAATTTAA |
| | | | GTTTATCTACTTTCTTATCAAAAAAAGATAAAGA |
| | | | AGATAGAGAGACTATAAAACTTTTTGAAAAAGCT |
| | | | TTTAAATTCGCTAGAAAACACAATACTTTAATTG |
| | | | TTGCAGCAGCTGGAAACCATGGATATGATATTTC |
| | | | AAATTCAAAAAAACTTGCACAACAACTAGGTAAA |
| | | | CCAAAGGACAATATCCTACTTCTTCCTGGTGGAG |
| | | | ATAACGATGTAATTACTGTATCTGCCACCACAGC |
| | | | ACAAAATACAAAAGCTTCCTACTCAAATTACGGG |
| | | | AAAACCATCTCGATTGCAGCTCCAGGTGGTGATC |
| | | | TTGACCCAAGTTTTTTGGATACAAATAAGGTTGA |
| | | | TCTATATAGCTTGGTATGGACTACTTCCCCTGTA |
| | | | AATATCCCGCAAACTCCGCTAAGTAAGTCACTGG |
| | | | GCTTTCAAAAGGGATATGAACCTACTTTAGGGAC |
| | | | AAGTGTAGCAGCTCCAATAGTAACTGCTACAGCA |
| | | | GCCCTATTAAAAACAGAATACTACGAAAAATACG |
| | | | GAGTGGAGATGTCAAATCGCAAAGTTGAGAAAAA |
| | | | CTTATATCAAAACACAGACCACTTTGAAAACTTA |
| | | | AATAAGAATGAAGTAGGTAGCGGTATCGTAAATG |
| | | | CTTACGAATCTTTGCTTAATATTGAGAGATAA |
| Seq. | class III - gene | B. velezensis | ATGAATAACGTATTAGCATTACAACAATTAAATA |
| ID. 67 | | | AAAGTGCTGAGGTGGAACCGCAAGTGGGACCAAC |
| | | | GATAACAACTATTACAGTAATACCTACAATGGTT |
| | | | ATATCCTTAATAAGCAATTATTGTGACAATCATG |
| | | | CTTCATAA |
| Seq. | class III lanthipeptide - | B. velezensis | ATGAGAGATGTATTGGAACTTCAACAACTGAATG |
| ID. 68 | gene | | AAGGAACTGAAGTAGAAGCTTCAAAAGGCAAAGG |
| | | | CGGCTTACTTACACTGATCACAACGGTAGTGGGC |
| | | | ACATCTACTATTAGCAACAACTGCGGATAA |

3. In Vitro Inhibition of E. amylovora (CM1) Growth in Solid Dual-Plate Assay and Antibiogram

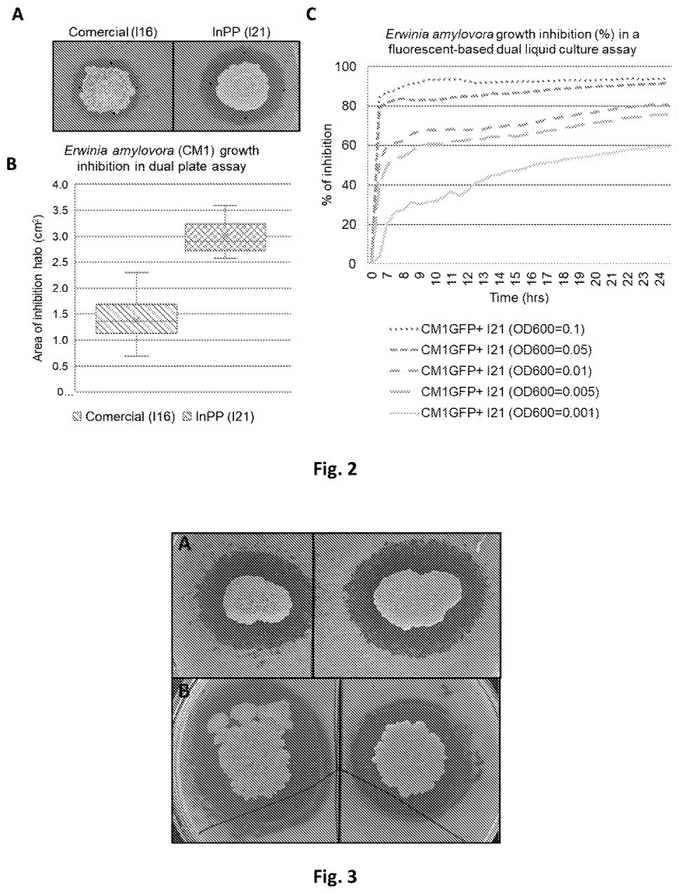

In an embodiment, dual-culture inhibition plate assays were tested in KB agar plates. Single colonies from CM1 and the two Bacillus strains, I21 and Commercial (I16), were incubated overnight in KB liquid media with linear shaking at 28° C. The bacterial concentration of each isolate was verified by OD₆₀₀. The E. amylovora (CM1) inoculum was adjusted to OD₆₀₀=0.1 and 100 μL of the inoculum were spread onto KB agar plates. The plates were allowed to dry for 20 min after which 10 μL of each Bacillus strain with an OD₆₀₀=0.5 were spotted on top of the inoculated pathogen. In the case of the antibiogram, the procedure was as above antibiotic discs embedded in 15 μl of different Streptomycin concentrations (50, 100, 500, 2500 and 1000 μg/ml) and placed. Data collection was also performed 2 days post inoculation (2 DPI). FIG. 2 shows the inhibition assay results based on dual-culture of CM1 with I21 and Commercial (I16). I21 displayed significantly greater in vitro inhibition efficiency than Commercial (I16) as quantified in FIG. 2.

In an embodiment, dual-plate inhibition assays were also performed with two E. amylovora reference strains with Serenade ASO (used as control) and 121. This was essential to display that the 121

B. velezensis is active against different E. amylovora strains including those isolated from different countries and different plant species. As can be seen from FIG. 3, inhibition of E. amylovora CFBP 1430 (a virulent strain isolated in France/Belgium from a Crataegus sp plant; (Paulin, 1973) and E. amylovora Ea273 (ATCC 49946; a virulent strain isolated from an infected apple tree in New York State; (Sebeihia, 2010) was observed for Commercial (I16) (left panel) and I21 (right panel).

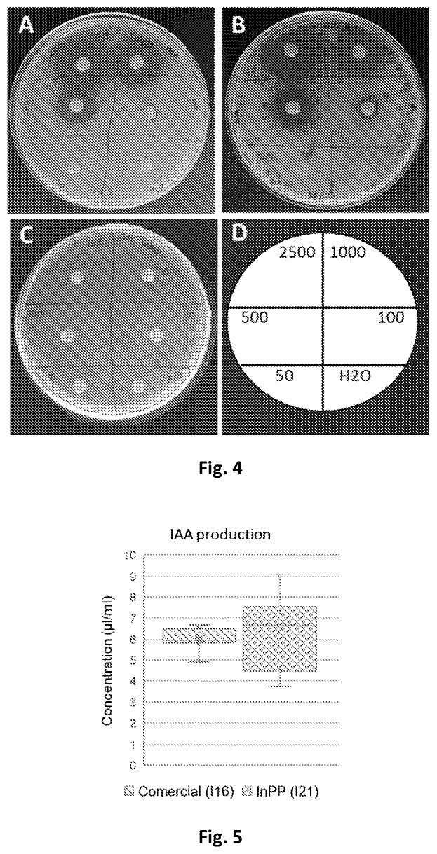

FIG. 4 presents the antibiogram of different E. amylovora strains with streptomycin, a potent antibiotic. Whilst E. amylovora CFBP 1430 and E. amylovora Ea273 (ATCC 49946) were highly susceptible towards the antibiotic effect, E. amylovora CM1, the Portuguese isolate from Campo Maior was completely resistant. This displays that the I21 B. velezensis strain is surprisingly active against a strain of E. amylovora that is highly resistant to strong antibiotics.

4. In Vitro Inhibition of E. amylovora (CM1) Growth in Liquid Dual-Plate Assay.

In an embodiment, E. amylovora (CM1) was electroporated using a described protocol (Spinelli, 2005) and transformants were selected in KB agar supplemented with tetracycline (10 μg/ml). A transformation efficiency of 1.18×10⁴ CFU/μg was obtained. E. amylovora (CM1) was transformed with pHC60 a broad host range plasmid (Cheng et al., 1998) that constitutively expresses GFP. To prepare CM1 competent cells, the bacterium was grown in LB overnight at 28° C. with linear shaking at 120 rpm. The culture was diluted to an OD₆₀₀ of 0.1 with fresh LB. When the OD₆₀₀ reached 0.8, cells were kept on ice for 20 min. A volume of 12 mL was centrifuged at 8,000×g for 5 min at 4° C. and the supernatant discarded. The pellet was washed in the same original volume with ice-cold sterile MilliQ water. The resuspended pellet was centrifuged at 8,000×g for 5 min and the procedure repeated twice, decreasing the water volume by half. Finally, the pellet re-suspended in 250 μl of ice-cold sterile MilliQ water. The transformation was performed using 100 μL of the electrocompetent cells. The plasmid DNA was purified with NZYGelpure kit and 550 ng were used for the transformation. A 2 mm gap electroporation cuvette was used on a Gene Pulser Xcell™ Electroporation System (BIORAD, Amadora) with a pulse of 800 Ohms, 2.5 kV and 2.5 pFD. The recovery was done by immediately adding 1 ml of SOC medium followed by incubation at 28° C. with minimal shaking for 1 hr. Transformed bacteria was spread on LB agar plates supplemented with 10 μg/mL tetracycline. One day after incubation, positive transformants were confirmed by PCR using the same program set up for 16S rRNA amplification but with the alternative primers M13Fw/M13Rev (M13Fw-CACGACGTTGTAAAACGACGGCCAGTG (SEQ ID No. 71); M13Rev-GTGAGCGGATAACAATTTCACACAGG (SEQ ID No. 72)), targeting the GFP expression plasmid. GFP expression was confirmed observing the colonies under a UV transilluminator and fluorescent light microscope. E amylovora (CM1) transformed with GFP is herein termed CM1GFP The in vitro dual liquid culture inhibition assay between CM1 and I21 was set up in 96-well plates, with a total volume of 200 μl in KB media. E. amylovora (CM1) OD₆₀₀ was grown overnight in KB and an exponential growing culture was diluted to OD₆₀₀ 0.01. Several InPP (I21) OD₆₀₀ were tested, namely 0.1, 0.05, 0.001, 0.0005, 0.0001 and 0 (control). Each condition was repeated in triplicate. CM1GFP growth was assessed by fluorescence measurement at 488 nm every 30 min, during 24 h using a BioTek Synergy HTX plate reader. FIG. 2 shows inhibition assay results based on dual-culture of CM1 with I21 and Commercial (I16).

### 5. Indole-Acetic-Acid (IAA) Production

Auxins are hormones that enhance plant root growth, hence improving crop growth and productivity. Microbes that produce auxin can help plants improve biotic and abiotic stress tolerance. FIG. 5 shows the quantification of auxin production of I21 and Commercial (I16). In an embodiment, the methodology used for auxin quantification was an adapted colorimetric procedure (Gilbert, 2018). Commercial (I16) and I21 were incubated in liquid Luria Broth (LB) media at 28° C. with 180 rpm during 16 h. The OD₆₀₀ was then adjusted to 0.05 and the inoculum supplemented with L-tryptophan (5 mg/mL). A volume of 200 μL of inoculum was added onto a 96-well plate and incubated at 28° C. with 180 rpm for 24 h. Cultures were centrifuged at 3,000 rpm for 30 min and 100 μL of supernatant were transferred into a fresh 96-well plate. A 1:1 (v/v) solution of Salkowski's reagent (34.3% (v/v) perchloric acid and 10 mM FeCl₃) was added to each well and the plate incubated for 30 min at room temperature and protected from light. The change in colour was recorded at 530 nm in a spectrophotometer. An indole-acetic-acid (IAA) standard curve was produced in a range of (1-0 μg/mL). Sterile LB supplemented with 5 mg/mL L-tryptophan was used as control and its relative IAA concentration was subtracted from those obtained for both bacterial isolates. The amount of IAA produced was obtained by comparison with the IAA standard curve. Commercial (I16) was used as positive control for IAA production.

### 6. 1-Aminocyclopropane-1-Carboxylic Acid (ACC) Deaminase Activity.

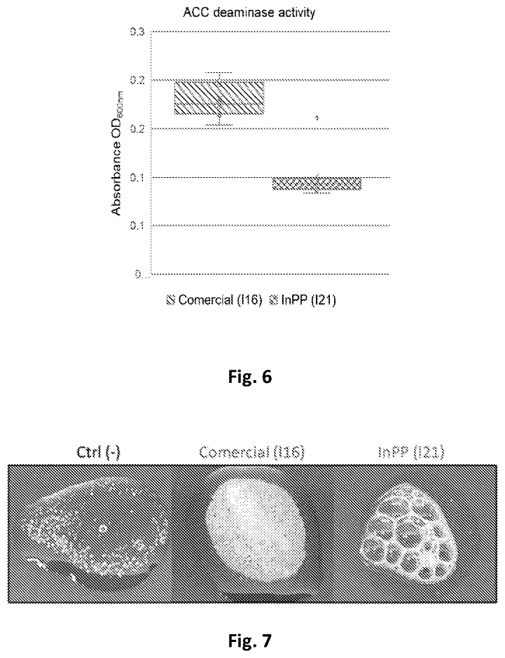

Ethylene is a hormone that mediates plant response to environmental stressors. However, when produced above a certain threshold, ethylene hinders plant growth and development. Bacteria that produce 1-aminocyclopropane-1-carboxylate deaminase (ACCd), an enzyme that breaks down an intermediate of ethylene, have the ability to ameliorate the deleterious effects of environmental stressors. FIG. 6 shows the quantification of ACC deaminase activity of I21 compared with the Commercial (I16). The qualitative assessment of ACC deaminase activity was based on previously developed methodology (Penrose, 2003). Commercial (I16) and I21 were incubated at 28° C., 180 rpm in liquid LB for 16 h. Pre-inoculums were adjusted to an OD₆₀₀ of 0.05 in minimal nutrient medium M9 using ACC as sole nitrogen and carbon source (22 mM KH₂PO₄, 33.7 mM Na₂HPO₄, 9.35 mM NH₄Cl, 8.55 mM NaCl, 1 mM MgSO₄, 0.3 mM CaCl₂) and 3 mM ACC). A volume of 100 μL of each inoculum was added to a 96-well plate and incubated for 48 h. Bacterial growth indicates ACCd activity and was recorded by spectrophotometric measurement at 600 nm (OD₆₀₀). Commercial (I16) was used as positive control. A blank control was also used to normalise the data. Bacterial growth, as measured by an increase in optical density measurement at 600 nm over time, is considered to confirm ACC deaminase activity.

### 7. Catalase Activity.

Catalase is an enzyme that helps in the breakdown of hydrogen peroxide (H₂O₂), a toxic compound that can damage cells by producing reactive oxygen species (ROS), into water and oxygen. Bacteria producing catalase are well adapted to harsh environments since they can detoxify their environment by breaking down the H₂O₂ produced by pathogen bacteria, not only protecting themselves and other organisms from its harmful effects but also causing the inhibition of the pathogen's growth and reduction of their virulence. Besides they can also help to promote plant growth. In an embodiment, qualitative catalase activity was evaluated by adding fresh single colonies of 121, Commercial (I16) and a bacterial negative control from an LB agar plate onto a solution of 3% (v/v) hydrogen peroxide on a sterile slide (Hansen, 1978). Oxygen release by means of effervescence indicates positive catalase activity as shown (FIG. 7) for I21 and to a lesser extent for Commercial (I16).

8. E. amylovora Infectivity in Detached Pear Assays.

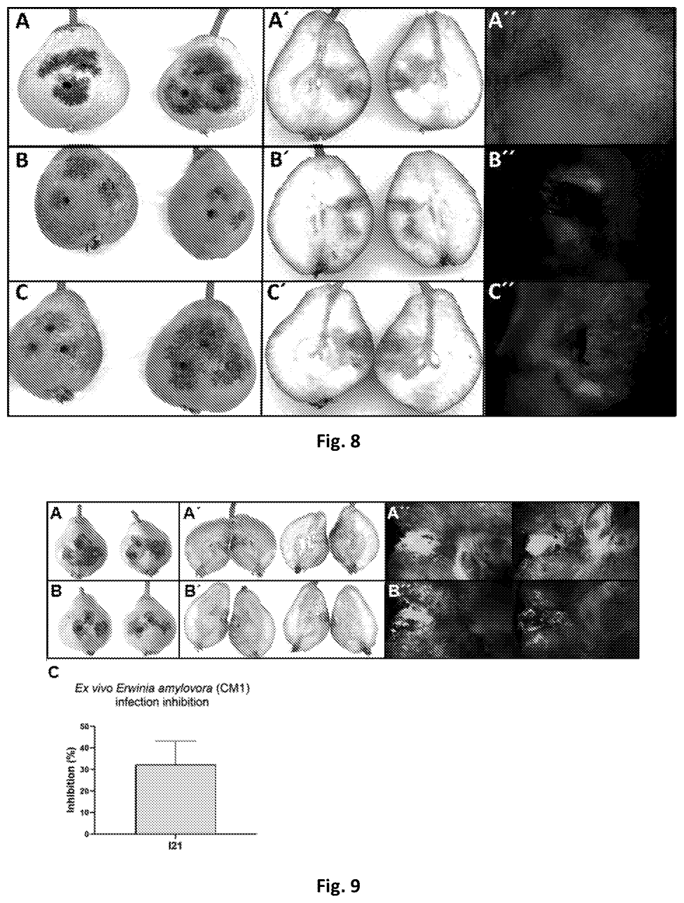

In an embodiment, Portuguese Rocha immature pears of caliber 55/60 mm were used as hosts to test the biological control capacity of I21 against E. amylovora (CM1GFP) infection. Two inoculation tests were performed according to a previously descried procedure (Donat, 2005) with some modifications. E. amylovora (CM1GFP) was grown in liquid LB media with linear shaking at 120 rpm for 48 h. The growth was assessed by means of OD₆₀₀ measurement. The bacterial cells were centrifuged at 5,000×g for 5 min and the pellet resuspended to 10⁹ CFU/mL in 1× phosphate buffered saline (PBS, pH=7.4) which corresponds approximately to an OD₆₀₀ of 1. The Bacillus strains were freshly grown from a single colony in LB media to OD₆₀₀ of 1, pelleted as above and re-suspended to an OD₆₀₀ of 1 in 1×PBS. Just before inoculation, the immature pears were surface sterilised by sequential submersion in 10% (v/v) bleach for 2 min, sterile distilled water for 2 min, then sprayed with 70% (v/v) ethanol and air dried in a sterile hood for 5 min. The pears were pierced with a needleless syringe in three vertices of a triangle to create inoculation holes which were filled at the incision site with 20 μl bacterial solutions containing a 1:1 mixture of combinations of E. amylovora (CM1GFP) and each Bacillus strain and the respective controls of each Bacillus strain and PBS, and E. amylovora (CM1GFP) and PBS. The bacterial solutions were allowed to penetrate into the holes for 5 min, after which the pears were placed on wet filter paper in a plastic container to maintain a humid environment and kept at 25° C. for 5-7 days. Each experimental group contained 6 pears and experiments were repeated three times. Images of pears from all groups are displayed in FIGS. 8 and 9.

In the in vitro dual plate assay in solid media, CM1 inhibition halo is twice the size in the presence of I21 B. velezensis when compared to the Commercial (I16) (FIG. 2, Panels A and B). In the in vitro assay in liquid media CM1 was inhibited by I21 by around 50% at an OD₆₀₀ as low as 0.001, whereas at OD₆₀₀ of 0.1 there is almost complete inhibition (FIG. 2, Panel C).

In an embodiment, I21 displayed a higher capacity to produce the hormone auxin, known to improve plant biotic and abiotic stress tolerance that helps plant development, than the Commercial (I16) (FIG. 5).

In an embodiment, the disclosed I21 strain, like Commercial (I16), displayed deaminase activity (FIG. 6).

In an embodiment, I21 showed higher catalase activity than Commercial (I16) as observed by stronger bubble production resulting from higher release of oxygen (FIG. 7). This result suggests that I21 has greater chance of survival in harsh environments because of its inherent capacity for auto-protection against oxidative stress than the Commercial sample (I16).

In detached pear infectivity assays, I21 and Commercial (I16) were tested as potential biological control agents against E. amylovora (CM1-GFP) causing fire blight in fruit (FIGS. 8 and 9). When CM1GFP was inoculated alone, typical fire blight disease symptoms were observed, soon after infection the skin turning slightly off-colour and quickly turning to brown. However, when InPP (I21), at an OD₆₀₀ of 0.1, was co-inoculated with CM1GFP, the symptoms observed were clearly less severe than CM1GFP alone. This reduction in disease symptoms is clearly more substantial than that observed for treatment with Commercial (I16) at the same OD. These results were further corroborated by observation of the pear slices under UV light on a stereomicroscope, in which there was a very clear reduction of CM1GFP expression in pears treated with 121+CM1GFP in comparison to either CM1GFP alone or I16+CM1GFP. Together, these results demonstrate that surprisingly in detached pear assays, the disclosed I21 has a stronger capacity to reduce E. amylovora growth and fire blight symptoms than Commercial (I16).

9. I21 B. velezensis Suspension Concentrate Formulation.

In an embodiment, to an aqueous solution of sodium chloride (0.9% w/v, 80 mL) at room temperature it was added an aqueous solution of alginate (3% w/v, 53 mL) followed by Silwett-77 (0.2 mL), with stirring. A pellet of I21 (2.04×10⁹ CFU/mL) was suspended in LB medium (27 mL) that was added to aqueous solution of sodium chloride (0.9% w/v, 133 mL). This mixture was added to the initial alginate-containing solution with gentle agitation and diluted with water to a final volume of 15-16 L, resulting in the I21 B. velezensis formulation.

### 10. Alginate Microparticles.

In an embodiment, an aqueous solution of sodium alginate (0.5% w/v, 5 mL) was sprayed onto an aqueous solution of calcium chloride (CaCl₂)) (20% w/v, 14 mL) and tween 80 (15 μL) with continuous agitation at 200 rpm at room temperature, in aliquots of 0.5 mL at the rate of 38 L/min. At the end of the process, excess CaCl₂) was eliminated and the pelleted microparticles (average size 50-100 μm) were recovered in 1 mL of deionized sterile water using a vortex.

11. Alginate Microparticles Loaded with I21 B. velezensis.

In an embodiment, a pellet of I21 at OD₆₀₀=0.6 (10⁸ CFU/mL, 3 mL) was suspended in 200 μL of deionized sterile water and the bacterial suspension was added to an aqueous solution of sodium alginate (0.5% w/v, 5 mL). The mix was sprayed as described above to produce microparticles loaded with bacteria by crosslinking with CaCl₂) (20%, 14 mL).

12. In Vitro Formulated I21 B. velezensis Efficacy Studies.
E. amylovora Growth Inhibition Assay

In an embodiment, E. amylovora was grown in LB medium at 28° C. with agitation at 180 rpm, then mixed with 50% (v/v) glycerol at a 1:1 ratio (v/v). E. amylovora stocks were kept frozen at −80° C.

In an embodiment, E. amylovora was transferred from the frozen stock to a Ø 90 mm plate of KB medium and grown overnight at 28° C. and re-streaked to a fresh KB petri-dish and grown for 48 h in the same conditions. The plates were kept at 4° C. for up to two weeks.

In an embodiment, E. amylovora was transferred from the 4° C. stock to LB medium and grown for several hours at 28° C. at 180 rpm. The OD₆₀₀ of the cultured I21 bacteria was adjusted to 0.01, from which 100 μL were spread onto KB agar Ø 90 mm plates and left to dry. A 10 μL drop of test-sample was pipetted into the centre of the plate and left to dry. The plate was sealed with parafilm and incubated for 3 days at 28° C. The plates were photographed and the inhibitory area quantified using image J software (java 1.8.0_172). Briefly, a scale was set considering the diameter of the petri dish (bottom plate), the total area comprising the test-sample and inhibition zone was highlighted, and automatically quantified in cm². The same was done for the area of the test-sample alone. The final inhibited area calculated as follows:

$$Inhibition ( {cm}^{2} ) = Total area ( inhibition zone area + test - sample growth area ) / Test - sample growth area .$$

The experiment was repeated as described above with the disclosed I21 strain initially cultured at an OD of 1.0.

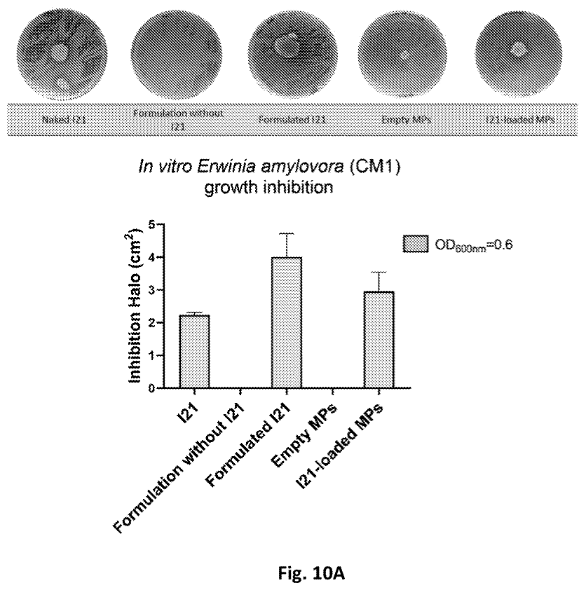

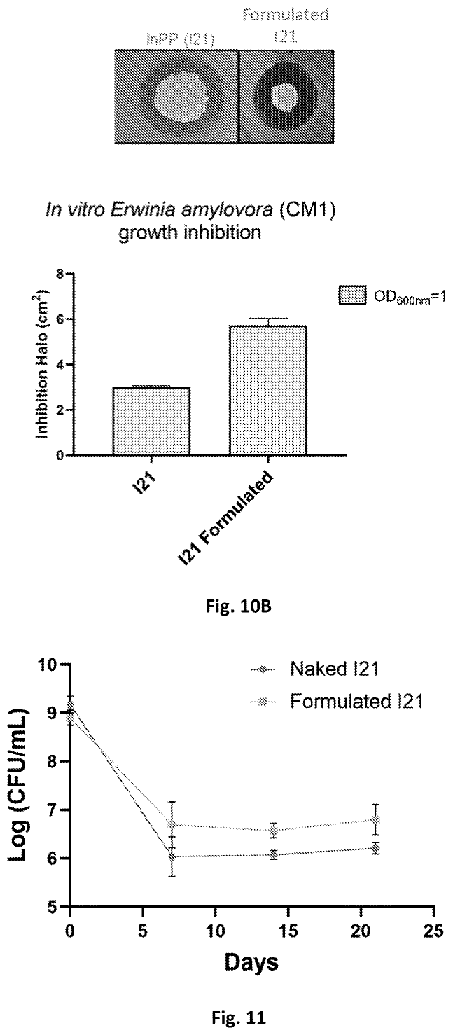

Results are shown in FIG. 10A and FIG. 10B. As expected, neither the suspension concentrate formulation without I21 nor the unloaded alginate microcapsules alone, inhibited E. amylovora. However, the I21 suspension concentrate formulation and the I21-loaded alginate microcapsules showed a greater inhibition of E. amylovora than that of non-formulated I21 alone.

Another advantage of the I21 suspension concentrate formulation can be seen in FIG. 11. Over a 21-day period, under storage at 4° C. and protected from light, the viability of the formulated I21 is greater than that of the naked I21 at all timepoints tested.

13. Ex Vivo Microencapsulated I21 B. velezensis Efficacy Study in Pears

In an embodiment, for the evaluation of the I21 effect against E. amylovora in pears, as well as the formulations containing the bacterium, detached pear assays were performed. Plastic containers (30 cm×20 cm) were sterilized for 10 min under UV light. Immature pears caliber 55/60 mm (were washed for 2 min in 10% bleach, rinsed in distilled H₂O for 2 min and sterilized with 70% ethanol. In a flow chamber, 3 holes were made in each fruit with a needleless plastic syringe followed by the application of each treatment (10 μL per hole). Pears inoculated with the treatments were placed in the sterile container over a paper sheet moistened with 20 mL of sterile H₂O. Boxes were closed with parafilm and incubated for 24 h at 25° C. After this period 10 μL of CM1GFP E. amylovora in PBS (OD₆₀₀ nm=0.5) were added to each hole, the boxes closed with parafilm and incubated for 25° C. for 5 days. Pears were photographed and cut in slices so that the holes sections could be photographed under fluorescence stereomicroscope (Nikon). Images were taken of all 6 pears per experiment under the exact same magnification, zoom, camara and UV settings and were quantified using and automated macro on ImageJ.

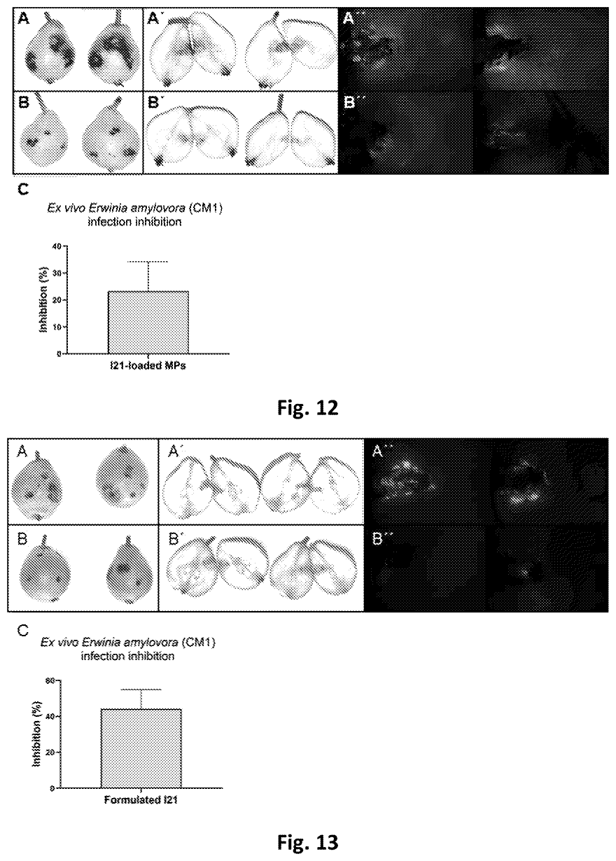

Results are shown in FIG. 12. As expected, the unloaded alginate microcapsules alone exhibited little effect. However, the I21-loaded alginate microcapsules provided inhibition of E. amylovora greater than that of non-formulated I21 alone.

14. Ex Vivo Formulated I21 B. velezensis Efficacy Study in Pears.

In an embodiment, the same procedure as above was applied with formulated I21 and the results are displayed in FIG. 13. The formulated I21 provided inhibition of E. amylovora similar than that of non-formulated 121.

### 15. Spray-Drying Formulation of 121.

In an embodiment, bacteria was grown in LB medium, and a pellet was recuperated from 10 ml of inoculum at OD₆₀₀=0.6 (exponential phase), and after 48 h of growth (stationary phase) at 28° C. at 180 rpm. An aqueous solution of filtered MgSO₄ (10% w/v) was prepared and used to resuspend both pellets in a final volume of 100 mL each. The control consisted in 100 ml of bacteria grown in LB medium for 48 h and sprayed directly without pelleting. The initial bacterial concentration was determined using the spread plating technique for CFU counts by collecting 1 mL from each solution before spray drying (without affecting the final volume).

In an embodiment, a laboratory-scale Mini spray dryer was used and prepared by setting the inlet T° to 120° C., the outlet T° to 80° C., the feed rate of the pump at 10% (^{˜}3 mL/min), the air flow rate at 40 mm (^{˜}600 l/h), and the aspirator at 100%. Each sample was fed to the drying chamber and the final powder was collected with a spatula and transferred into a 15 mL falcon tube. The tube was weighed to assess the final yield, and samples were stored at 4° C. until use.

In an embodiment for bacterial quantification for viability results, spread plating technique for colony counts was performed for all treatments on nutrient agar (23 g/L) plates. 100 μl of multiple 10-fold serial dilutions were plated and the plates were kept for 48 hours in an incubator at 28° C. The colony counting was performed by choosing an appropriate dilution (between 10 and 50 colonies per plate) and the bacterial loss during the spray drying process was assessed in percentage using the following formula:

$$S = \frac{CFU counts in total recuperated grams of powder}{initial CFU counts of bacteria in the total sprayed volume} \times 100$$

In an embodiment, of the in vitro assay against E. amylovora, the spray-dried samples were stored at 4° C. for two months. All samples were rehydrated in PBS buffer 1× and were applied in a growth inhibition assay of E. amylovora in vitro as previously described. The control consisted in bacteria grown on the day of the experiment to an OD₆₀₀=1.2 (stationary phase of growth).

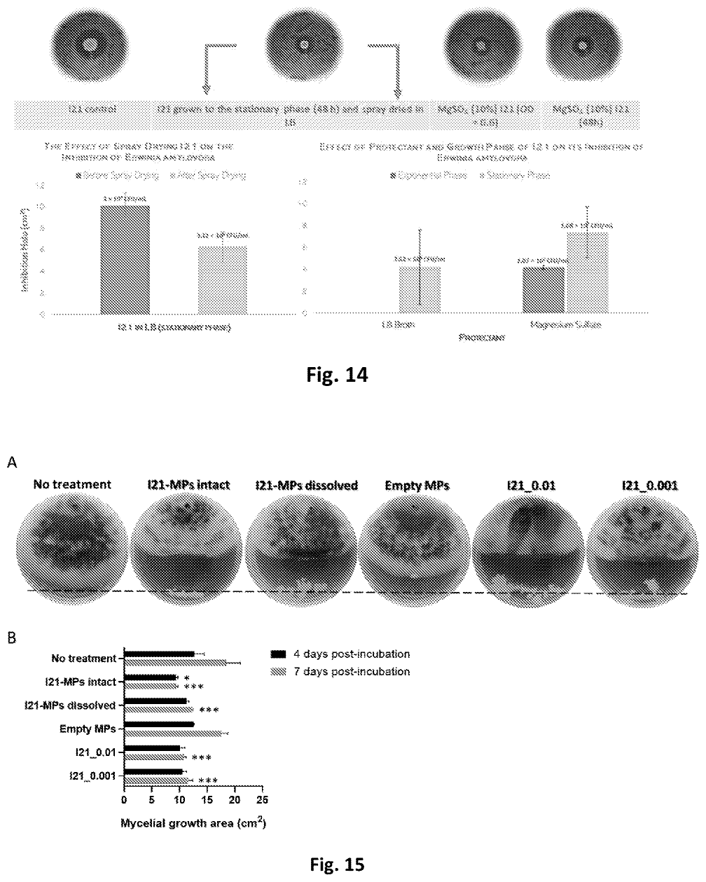

Results are presented in FIG. 14. Spray-dried formulations were particularly effective in vitro against growth of E. amylovora.

16. In Vitro Inhibition of M. oryzae Growth

In an embodiment, the in vitro antifungal activity of the naked I21 and formulated I21 was assessed on the basis of mycelial growth inhibition of M. oryzae isolate M22.7 (isolated and identified by PCR at InnovPlantProtect facilities). Briefly, actively growing M. orzyae was transferred with a 7 mm diameter cork-borer to potato-dextrose agar plates, distanced 5 mm from the extremity. M. oryzae was left to grow for 5 days at 25° C., and then 5 μl of naked I21 (collected at the exponential phase, OD 600=0.6) and I21-loaded alginate microparticles were incubated in the opposite extremity, also distanced 5 mm from the outer surface. The test-samples were left incubated at 25° C. for 7 days, and a negative control was included. Antifungal activity was defined by the area of M. oryzae growth, using imageJ software (java 1.8.0_172).

In an embodiment, I21 was tested against M. oryzae in naked and encapsulated forms. I21-loaded alginate microparticles displayed remarkable mycelial growth inhibitory capacity, being comparable to naked I21 (I21_0.01 and 121_0.001, FIG. 15). Thus, I21 activity is not compromised by the encapsulation process. Empty microparticles exhibited a non-statistically significant inhibitory effect. Furthermore, only the control and unloaded MP groups permitted further growth of M. orzyae between 4 and 7-days whilst all active-containing treatment groups were able to prevent further fungal expansion.

17. M. oryzae In Vitro Growth Inhibition by I21 B. velezensis by GFP Fluorescence Detection

In an embodiment, spore (conidia) from the GFP-expressing Portuguese isolate M. orzyae PR9GFP were harvested and tested at a concentration of 10³ spores/mL in a black, flat-bottom 96-well plate in the presence of I21 at three different OD₆₀₀ concentrations, in a 10% volume ratio of I21 cells per well. Fluorescent readings were taken for GFP signal (Excitation: 479 nm, Emission: 520 nm) and measurements were taken every 30 minutes for 72 h at 25° C., under continuous orbital shaking at 282 cycles per minute, using a Synergy H1 multi-well plate reader (BioTek).

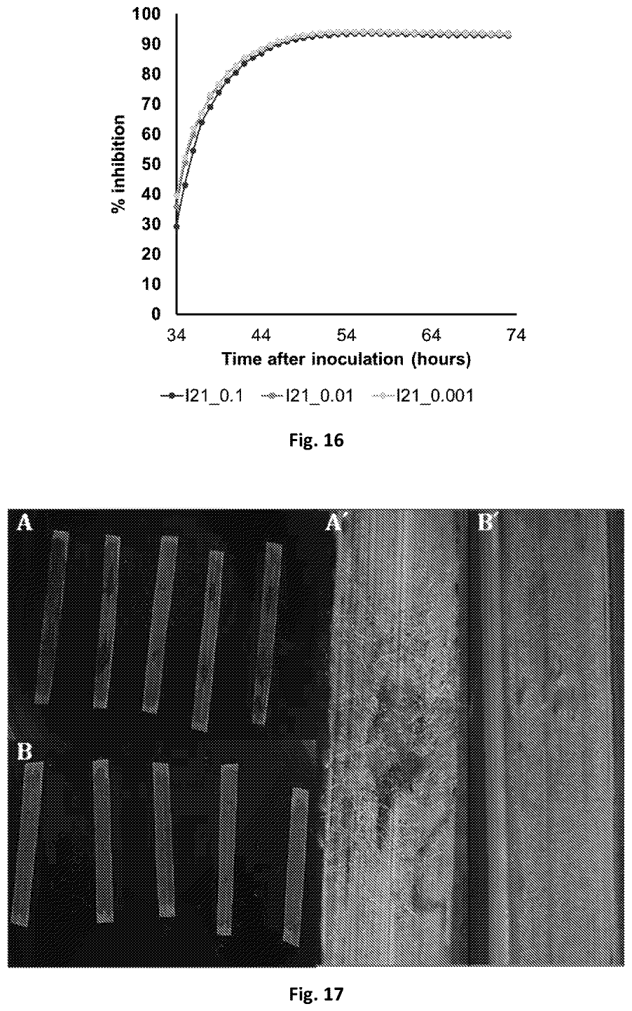

As can be seen from FIG. 16, all concentrations of I21 were highly effective at inhibiting growth of M. orzyae with inhibition rates close to 100%.

18. M. oryzae Ex Vivo Growth Inhibition by I21 B. velezensis on Detached Presto Leaves.

In an embodiment, M. oryzae M22.7 cultures were grown on rice flour for 3 days in the dark and 3 days under constant light (50/50 white light and purple light). Spores were harvested by scraping the cultures with 0.02% Tween-20 and counting was done with a Neubauer chamber.

In an embodiment, rice leaves from 3 to 4-week old Presto plants were cut and sterilized with 70% EtOH and washed with sterile water. Fresh single colonies were grown in liquid LB media. 5 ml of inoculum was centrifuged at 5000 rpm for 10 min at room temperature and the pellet resuspended in the same initial volume of 1×PBS supplemented with 0.25% gelatin. Rice leaves were submerged into the bacterial solution and the surplus removed using a sterile filter paper. Submerged leaves were placed on 0.7% agar plates with 2 ppm 6-benzylaminopurine overnight in the dark. Next day, 2.5 μL of M22.7 at 10⁶ conidia/mL were spotted on top of the leaves. Wounds were made by pricking the inoculated points with a sterile needle. Leaves were placed overnight in the dark then incubated for 6/7 d with a 16 h/8 h photoperiod.

As can be observed from the data presented in FIG. 17, I21 was particularly effective at controlling blast disease symptoms caused by M. orzyae in this assay.

Taking the results from each of the experiments in the Examples recited above, it is now surprisingly possible to provide an efficacious, standalone formulation of a BPPA that confers superiority over an existing commercial product to control fire blight caused by the pathogen E. amylovora. The I21 B. velezensis strain disclosed herein according to the present invention may be used for control of additional plant diseases caused by other pathogens belonging to the Erwinia genus. Moreover, the I21 B. velezensis strain of the present disclosure allows for control of fire blight and to increase plant resistance by using I21 B. velezensis strain, therefore making an unexpected contribution to the safe and sustainable production of pome fruits and ornamental plants. Additionally, the disclosed I21 B. velezensis can also be used to control other plant diseases such as rice blast caused by the fungus M. oryzae. The B. velezensis (I21) strain disclosed herein according to the present invention may be used for control of additional plant diseases caused by other fungi besides those belonging to the Magnaporthe genus. Moreover, the present invention allows for control of rice blast and to increase plant resistance by using B. velezensis (I21) strain, therefore making an unexpected contribution to the safe and sustainable production of rice.

The term ‘comprising’ whenever used in this document is intended to indicate the presence of stated features, integers, steps or components, but not to preclude the presence or addition of one or more other features, integers, steps, components or groups thereof.

All references recited in this document are incorporated herein in their entirety by reference, as if each and every reference had been incorporated by reference individually.

Those skilled in the art will recognise or be able to ascertain using no more than routine experimentation, many equivalents to the specific embodiments of the invention described herein. The scope of the present invention is not intended to be limited to the above description, but rather is as set forth in the appended claims.

Where singular forms of elements or features are used in the specification of the claims, the plural form is also included, and vice-versa, if not specifically excluded. For example, the term ‘a bacterium’ or ‘the bacterium’ also includes the plural forms ‘bacteria’ or ‘the bacteria’, and vice versa. In the claims, articles such as ‘a’, ‘an’, and ‘the’ may mean one or more than one unless indicated to the contrary or otherwise evident from the context. Claims or descriptions that include ‘or’ between one or more members of a group are considered satisfied if one, more than one, or all the group members are present in, employed in or otherwise relevant to a given product or process unless indicated to the contrary or otherwise evident from the context. The invention includes embodiments in which exactly one member of the group is present in, employed in or otherwise relevant to a given product or process. The invention also included embodiments in which more than one or all of the group members are present in, employed in or otherwise relevant to a given product or process.

Furthermore, it is to be understood that the invention encompasses all variations, combinations and permutations in which one or more limitations, elements, clauses, descriptive terms, etc., from one or more of the claims or from relevant portions of the description is introduced into another claim. For example, any claim that is dependent on another claim can be modified to include one or more limitations found in any other claim that is dependent on the same base claim. Furthermore, when the claims recite a composition, it is to be understood that methods of using the composition for any of the purposes disclosed herein are included, and methods of making the composition according to any of the methods of making disclosed herein or other methods known in the art are included, unless otherwise indicated or unless it would be evident to one of ordinary skill in the art that a contradiction or inconsistency would arise.

Where ranges are given, endpoints are included. Furthermore, it is to be understood that unless otherwise indicated or otherwise evident from the context and/or the understanding of one of ordinary skill in the art, values that are expressed as ranges can assume any specific value within the stated ranges in different embodiments of the invention, to the tenth of the unit of the lower limit of the range, unless the context clearly dictates otherwise. It is also to be understood that unless otherwise indicated or otherwise evident from the context and/or the understanding of one of ordinary skill in the art, values that are expressed as ranges can assume any subrange within the given range, wherein the endpoints of the subrange are expressed to the same degree of accuracy as the tenth of the unit of the lower limit of the range.

In addition, it is to be understood that any particular embodiment of the present invention may be explicitly excluded from any one or all of the claims. Where ranges are given, any value within the range may be explicitly excluded from any one or all of the claims. Any embodiment, element, feature, application or aspect of the compositions and/or uses and methods of the invention can be excluded from any one or more of the claims. For purposes of brevity, all embodiments in which one or more elements, features, purposes or aspects are excluded are not set out forth explicitly herein.

The above-described embodiments are combinable.

The sequence listing is part of the description.

The following claims further set out particular embodiments of the disclosure.

### REFERENCES

Alenezi, F. N., Slama, H. B., Bouket, A. C., Cherif-Silini, H., Silini, A., Luptakova, L., Nowakowska, J. A., Oszako, T. & Belbahri, L. (2021) Bacillus velezensis: A Treasure House of Bioactive Compounds of Medicinal, Biocontrol and Environmental Importance. Forests, 12, 1714.
Alonge, M., Soyk, S., Ramakrishnan, S., Wang, X., Goodwin, S., Sedlazeck, F. J.& Schatz, M. C. (2019) RaGOO: fast and accurate reference-guided scaffolding of draft genomes. Genome Biology, 20(1), 1-17.
Altschul, S. F., Gish, W., Miller, W., Myers, E. W & Lipman, D. J. (1990) Basic local alignment search tool. J. Mol. Biol., 215(3), 403-410.
Armstrong, D. J., Azevedo, M. D., McPhail, K. L., Mills, D. I., Banowetz, G., Halgren, A. B, Jackson, M. D. & Thimmaiah, M. (2010) Control of Erwinia amylovora with vinylglycines and bacteria that produce vinylglycines. US 2010/0189798 A1.
Baym, M., Kryazhimskiy, S., Lieberman, T. D., Chung, H., Desai, M. M., & Kishony, R. (2015). Inexpensive multiplexed library preparation for megabase-sized genomes. PloS one, 10(5), e0128036.
Brower, W. (2011) Formulation and method for treating plants to control or suppress a plant pathogen. U.S. Pat. No. 7,906,131 B2.
Campanella, J. J., Bitincka, L. & Smalley, J. (2003) MatGAT: An application that generates similarity/identity matrices using protein or DNA sequences. BMC Bioinformatics 4, 29.
Cantalapiedra, C. P., Hernández-Plaza, A., Letunic, I., Bork, P., & Huerta-Cepas, J. (2021). eggNOG-mapper v2: functional annotation, orthology assignments, and domain prediction at the metagenomic scale. Molecular biology and evolution, 38(12), 5825-5829.
Chaumeil, P. A., Mussig, A. J., Hugenholtz, P., & Parks, D. H. (2022). GTDB-Tk v2: memory friendly classification with the genome taxonomy database. Bioinformatics, 38(23), 5315-5316.
Chen, X. H., Koumoutsi, A., Scholz, R., Eisenreich, A., Schneider, K., Heinemeyer, I., Morgenstern, B., Voss, B., Hess, W. R., Reva, O. et al. (2007) Comparative analysis of the complete genome sequence of the plant growth-promoting bacterium Bacillus amyloliquefaciens FZB42. Nat. Biotechnol., 25, 1007-1014.
Cheng, H.-P. & Walker, G. C. (1998) Succinoglycan is required for initiation and elongation of infection threads during nodulation of alfalfa by Rhizobium meliloti. Journal of Bacteriology, 180(19), 5183-5191. Dagher, F. & Deziel, E. (2022) Biological control agents against fire blight. U.S. Pat. No. 11,337,426 B2.
Donat, V., Biosca, E. G., Rico, A., Penalver, J., Borruel, M., Berra, D., Basterretxea, T., Murillo, J. & Lopez, M. M. (2005) Erwinia amylovora strains from outbreaks of fire blight in Spain: Phenotypic characteristics. Annals of Applied Biology, 146(1), 105-114.
Elert E. Rice by the numbers: A good grain. Nature, 2014, 514, S50-S51.
Fan, B., Wang, C., Song, X., Ding, X., Wu, L., Wu, H., Gao, X. & Borriss, R. (2018) Bacillus velezensis FZB42 in 2018: The Gram-Positive Model Strain for Plant Growth Promotion and Biocontrol. Frontiers in Microbiology, 9, 2491.
Gharsa, H. B., Bouri, M., Mougou, H. A., Schuster, C., Leclerque, A. & Rhouma, A. (2021) Bacillus velezensis strain MBY2, a potential agent for the management of crown gall disease. PLOS ONE 16(6): e0252823.
Gibson, D. G. et al., (2010) Creation of a Bacterial Cell Controlled by a Chemically Synthesized Genome. Science, 329, 52-56.
Gilbert, S., Xu, J., Acosta, K., Poulev, A., Lebeis, S. & Lam, E. (2018) Bacterial Production of Indole Related Compounds Reveals Their Role in Association Between Duckweeds and Endophytes. Frontiers in Chemistry, 6, 265.
Gupta, G. (2021) Recombinant peptide to treat fire blight. WO 2021/202476 A1.
Gurevich, A., Saveliev, V., Vyahhi, N., & Tesler, G. (2013). QUAST: quality assessment tool for genome assemblies. Bioinformatics, 29(8), 1072-1075.
Hansen, S. L., & Stewart, B. J. (1978) Slide Catalase: A Reliable Test for Differentiation and Presumptive Identification of Certain Clinically Significant Anaerobes. American Journal of Clinical Pathology, 69(1), 36-40.
Heins, S. D., Manker, D. C., Jiimenez, D. R., McCoy, R. J., Marrone, P. G. & Orjala, J. E. (2000) Strain of Bacillus for Controlling Plant Diseases and Corn Rootworm. U.S. Pat. No. 6,060,051B.
Jensen, S., Bergh, O., Enger, O. & Hjeltnes, B. (2002) Use of PCR-RFLP for genotyping 16S rRNA and characterizing bacteria cultured from halibut fry. Can. J. Microbiol., 48(5), 379-86.
Joly, P., Calteau, A., Wauquier, A., Dumas, R., Beuvin, M., Vallenet, D., Crovadore, J., Cochard, B., Lefort, F. & Berthon, J.-Y. (2021) From Strain Characterization to Field Authorization: Highlights on Bacillus velezensis Strain B25 Beneficial Properties for Plants and Its Activities on Phytopathogenic Fungi. Microorganisms, 9, 1924.
Kim, Y. S., Lee, Y., Cheon, W. et al. (2021) Characterization of Bacillus velezensis AK-0 as a biocontrol agent against apple bitter rot caused by Colletotrichum gloeosporioides. Sci. Rep. 11, 626.
Lee, M. D. (2019). GToTree: a user-friendly workflow for phylogenomics. Bioinformatics, 35(20), 4162-4164.
Mazzuchi, U., Luccesse, C. & Mazzuchi, A. (2014) Composition and method for preventing infections of vegetable tissues caused by Erwinia amylovora. WO 2014/177996 A1.
Mullen, B. & Mullen, L. L. (2011) Biological pest control mixture containing D-limonene and nutmeg oil. US 2011/0274677 A1.
Myo, E. M., Than, W. M., Win, T. & Khai, A. A. (2021) Evaluation of the symbiotic performance of tomato plants inoculated with Bacillus velezensis NKG-2 and challenged with Fusarium wilt pathogen. Archives of Phytopathology and Plant Protection, 54(17-18), 1391.
Needleman, S. B. & Wunsch, C. D. (1970) A general method applicable to the search for similarities in the amino acid sequence of two proteins. J. Mol. Biol., 48(3), 443-453.
Parks, D. H., Imelfort, M., Skennerton, C. T., Hugenholtz, P., & Tyson, G. W. (2015). CheckM: assessing the quality of microbial genomes recovered from isolates, single cells, and metagenomes. Genome research, 25(7), 1043-1055.
Paulin, J.-P. & Samson, R. (1973) Le feu bactérien en France. II.-caractères des souches d'Erwinia amylovora (Burril) Winslow et al. 1920, isolées du foyer franco-belge. Ann. Phytopathol. 5:389-397.
Pennisi E. Armed and dangerous. Science, 2010, 327, 804-805.
Penrose, D. M. & Glick, B. R. (2003) Methods for isolating and characterizing ACC deaminase-containing plant growth-promoting rhizobacteria. Physiologia Plantarum, 118(1), 10-15.
Perrin, A., & Rocha, E. P. (2021). PanACoTA: a modular tool for massive microbial comparative genomics. NAR genomics and bioinformatics, 3(1), Iqaa106.
Prjibelski, A., Antipov, D., Meleshko, D., Lapidus, A., & Korobeynikov, A. (2020). Using SPAdes de novo assembler. Current protocols in bioinformatics, 70(1), e102.
Pusey, P. L. (1999) Control of fire blight on pome fruit trees with Erwinia herbicola. U.S. Pat. No. 5,919,446 B2.
Reva, O. N., Swanevelder, D. Z. H., Mwita L. A., Mwakilili, A. D., Muzondiwa, D., Joubert, Monique., Chan, W. Y., Lutz, S., Ahrens C. H., Avdeeva, Lylia, V., Kharkhota M. A., Tibuhwa D., Lyantagaye, S., Vater, J., Borriss, R. & Meijer, J. (2019) Genetic, Epigenetic and Phenotypic Diversity of Four Bacillus velezensis Strains Used for Plant Protection or as Probiotics. Frontiers in Microbiology, 10, 2610.
Rooney, A. P., Price, N. P., Ehrhardt, C., Swezey, J. L. & Bannan, J. D. (2009) Phylogeny and molecular taxonomy of the Bacillus subtilis species complex and description of Bacillus subtilis subsp. Inaquosorum subsp. Nov. International Journal of Systematic and Evolutionary Microbiology, 59(10), 2429-2436.
Salvà-Serra, F., Gomila, M., Svensson-Stadler, L., Busquets, A., Jaén-Luchoro, D., Karlsson, R., & Moore, E. R. (2018). A protocol for extraction and purification of high-quality and quantity bacterial DNA applicable for genome sequencing: a modified version of the Marmur procedure.
Sebaihia, M, Bocsanczy, A. M., Biehl, B. S., Quail, M. A., Perna, N. T., Glasner, J. D., DeClerck, G. A., Cartinhour, S., Schneider, D. J., Bentley, S. D., Parkhill, J & Beer S. V. (2010) Complete genome sequence of the plant pathogen Erwinia amylovora strain ATCC 49946. J. Bacteriol., April; 192(7): 2020-1.
Seemann, T. (2014). Prokka: rapid prokaryotic genome annotation. Bioinformatics, 30(14), 2068-2069.
Shin, J.-H., Park, B.-S., Kim, H.-Y., Lee, K.-H. & Kim, K.-S. (2021) Antagonistic and Plant Growth-Promoting Effects of Bacillus velezensis BS1 Isolated from Rhizosphere Soil in a Pepper Field. The Plant Pathology Journal, 37(3), 307-314.
Skamnioti P and Gurr S J. Against the grain: safeguarding rice from rice blast disease. Trends Biotechnol., 2009, 27, 141-150.
Spinelli, F., Ciampolini, F., Cresti, M., Geider, K., & Costa, G. (2005) Influence of Stigmatic Morphology on Flower Colonization by Erwinia amylovora and Pantoea agglomerans. European Journal of Plant Pathology, 113(4), 395-405.
Taylor, R. K., Guilford, P. J., Clark, R. G., Hale, C. N. & Forster, R. L. S. (2001) Detection of Erwinia amylovora in plant material using novel polymerase chain reaction (PCR) primers. New Zealand Journal of Crop and Horticultural Science, 29(1), 35-43.
Umemura, K. & Hatakeyama, S. (2022) Agent for controlling scald disease and method for controlling scald disease. EP 4,018,831 A1.
Wang, S.-W., Herrera-Balandrano, D.D., Wang, Y.-X., Shi, X.-C., Chen, X., Jin, Y., Liu, F.-Q. & Laborda, P. (2022) Biocontrol Ability of the Bacillus amyloliquefaciens Group, B. amyloliquefaciens, B. velezensis, B. nakamurai, and B. siamensis, for the Management of Fungal Postharvest Diseases: A Review. Journal of Agricultural and Food Chemistry, 70(22), 6591.

1. A Bacillus velezensis strain with the deposit with number CECT30902 received on 12 Jul. 2023 at the Spanish Type Culture Collection (CECT).

2. A Bacillus velezensis strain wherein the Bacillus velezensis strain comprises the following genes SEQ ID No. 49 to SEQ ID No. 68.

3. A Bacillus velezensis strain with a genome comprising the sequence of SEQ ID No. 30 to SEQ ID No. 48.

4. (canceled)

5. The Bacillus velezensis strain of claim 1, wherein the strain controls plant or fruit diseases.

6. (canceled)

7. The Bacillus velezensis strain of claim 5, wherein the disease is a fungal and/or a bacterial infection.

8. The Bacillus velezensis strain of claim 7, wherein the bacterial infection is cause by a gram-negative bacteria.

9. The Bacillus velezensis strain of claim 5, wherein the plant is a woody plant.

10. The Bacillus velezensis strain of claim 7, wherein the infection is caused by an Erwinia bacterial strain.

11. The Bacillus velezensis strain of claim 5, wherein the plant is rice.

12. The Bacillus velezensis strain of claim 5, wherein the disease is a rice blast.

13. The Bacillus velezensis strain of claim 5, wherein the disease is caused by a M. orzyae infection.

14. A composition for plant protection comprising an effective amount of the Bacillus velezensis strain of claim 1, and a suitable additive.

15. The composition of claim 14, wherein the suitable additive is selected from a list consisting of: a carrier, a solvent, a buffer, a stabiliser; a preservative, an excipient, a surfactant, an adhesion agent, an emulsifier, osmoprotectant, sun protectant, wetting agent, and mixtures thereof.

16. The composition of claim 15, wherein the carrier is alginate, chitosan, ulvan, fucoidan, carrageenan, xanthan gum, gellan gum, gelatin, arabic gum, lignin, maltodextrin, zein, protamine sulphate, lecithin, gelatin matrices, methacrylate-modified lignin, methacrylated gellan gum, polyvinyl alcohol, low density polyethylene, poly(ε-caprolactone), polyethylene glycol or poly(lactic-co-glycolic acid), or combinations thereof.

17. The composition of claim 14, further comprising an agriculturally acceptable compound, wherein the agriculturally acceptable compound is selected from: plant strengtheners, growth promoters, nutrients, fertilisers, vitamins, minerals, and mixtures thereof.

18. The composition of claim 14, further comprising a biological plant protector agent.

19. The composition of claim 14, whereein the effective amount of B. velezensis strain in the composition ranges from 1.0×10³ CFU/mL to 1.0×10¹¹ CFU/mL.

20. The composition of claim 14, wherein the composition is in the form of wettable powders, granules, flowables, microencapsulations or nanoencapsulations.

21. (canceled)

22. (canceled)

23. The composition of claim 14, wherein the composition is a biological plant protection agent.

24. A method for protecting or treating plants and/or fruit from fungal or bacterial infections comprising applying an effective amount of the Bacillus velezensis strain of claim 1.
